(19) United States
(12) Patent Application Publication
(10) Pub. No.: US 2009/0117177 A1
(43) Pub. Date: May 7, 2009

(54) Echogenic microbubbles and microemulsions for ultrasound-enhanced nanoparticle-mediated delivery of agents

(76) Inventors: Natalia Rapoport, Sandy, UT (US); Zhonggao Gao, Salt Lake City, UT (US)

Correspondence Address: ALAN J. HOWARTH, P.O. BOX 1909, SANDY, UT, 84091-1909, US

(21) Appl. No.: 11/920,942

(22) PCT Filed: May 23, 2006
(86) PCT No.: PCT/US2006/020347

§ 371 Date: Nov. 21, 2007

Related U.S. Application Data
(60) Provisional application No. 60/683,829, filed on May 23, 2005.

(51) Int. Cl. A61K 9/127; A61K 33/24; A61P 35/00

(52) U.S. Cl. 424/450; 424/649

(57) ABSTRACT

Described are methods and compositions for treating tumors, such as drug-sensitive tumors, inoperable tumors, poorly vascularized tumors, and multidrug resistant tumors, by intravenous or direct intratumoral injection of compositions comprising microemulsions and polymeric micelle-encapsulated biologically active agents. The methods and compositions also include microemulsions converting into microbubbles in situ upon injection. The methods disclosed optionally including applying a micelle disruption method such as ultrasound. Also disclosed are methodologies of imaging administration of agents in tissues using streams of microemulsions which create microbubbles in situ upon injection. The methods and compositions also include enhancement of tumor treatment through use of microemulsions which create microbubbles in situ, upon injection, as cavitation nuclei. The methods and compositions are also useful in enhancing intracellular drug delivery.

### RELATED APPLICATION UNDER 35 U.S.C. § 119(e)

This application claims the benefit of U.S. Provisional Patent Application Ser. No. 60/683,829, filed May 23, 2005, for “ECHOGENIC MICROBUBBLES AND MICROEMULSIONS FOR ULTRASOUND-ENHANCED MICELLE-MEDIATED DELIVERY OF AGENTS,” the contents of the entirety of which are incorporated by this reference.

### STATEMENT REGARDING FEDERALLY SPONSORED RESEARCH OR DEVELOPMENT

Work described herein was supported by National Institute of Health Grant R01 # EB 1033. The United States government may have certain rights in the invention.

### TECHNICAL FIELD

This invention relates to biotechnology and medicine and more particularly to imaging and therapy of tumors through delivery of drug-loaded microbubbles mixed with drug loaded micelles or other nanoparticles to tumors by, for example, intravenous or intratumoral injection and optionally applying ultrasound.

### BACKGROUND

It has been observed for poorly vascularised tumors, drug-sensitive tumors, or multidrug resistance tumors, that intravenous, systemic injections of chemotherapeutic agents are not effective because the chemotherapeutic agent does not reach the entire tumor mass. For some tumor types, multidrug resistance is due to the action of protein pumps that cause drug efflux from the tumor cells; for other tumor types, a poor blood supply to the tumor volume results in insufficient drug delivery to the tumor cells.

“Polymeric micelles” are known to those of skill in the art. (See, e.g., U.S. Pat. No. 6,649,702, U.S. Pat. No. 6,338,859 and U.S. Pat. No. 6,623,729, the contents of each of which are herein incorporated by this reference). Polymeric micelles have a core-shell structure that protects the contents of the micelle during transportation to the target cell. “Micelles” are clusters of soap-like molecules that aggregate in aqueous solution. The soap-like molecules have a hydrophilic polar ionic head moiety and a “greasy” hydrophobic carbon chain tail. The polar heads of the molecules lie on the outside of a microsphere where they are solvated by water, and the organic tails bunch together on the inside of the micellar sphere. Polymeric micelles have been proposed as drug carriers or delivery vehicles. (See, Bader, H. et al., Angew. Makromol. Chem. 123/124:457-485, 1984). Polymeric micelles are efficient carriers of hydrophobic drugs because the hydrophobic moieties of the amphiphilic monomers solubilize the drugs, encapsulating the drug in the inner core of the micelle. Polymeric micelles offer various advantages such as a relatively small size (from tens of nanometers to hundreds of nanometers) and a propensity to evade scavenging by the reticuloendothelial system.

A similar amphiphilic chemical, lipoprotein, has also been proposed as a vehicle for the targeting and delivery of chemotherapeutic compounds because tumors express an enhanced need for low density lipoproteins (also known as “LDL”). However, the efficiency of lipoproteins as carriers has been questioned because drug-incorporated lipoproteins are also recognized by healthy cells, potentially resulting in delivery of the drug to the wrong cells, and because LDL would have to compete with natural lipoproteins for receptor sites on tumors.

Recent pharmaceutical research surrounding polymeric micelles has been focused on the potential use of copolymers having an A-B diblock structure wherein A represents the hydrophilic shell moiety and B represents the hydrophobic core moiety. Multiblock A-B-A copolymers such as poly(ethylene oxide)-poly(propylene oxide)-poly(ethylene oxide) (“PEO-PPO-PEO”) can also self-organize into micelles and have been described as potential drug carriers. (See, Kabanov, A. V. et al., FEBS Lett., 258:343-345, 1989). The hydrophobic core, consisting of a biodegradable polymer, such as a poly(β-benzyl-L-aspartate) (“PBLA”), poly(D,L-lactic acid) (“PDLLA”) or poly(ε-caprolactone) (“PCL”), serves as a reservoir for a hydrophobic drug, protecting it from contact with the aqueous environment. The core may also consist of a water-soluble, hydrophilic polymer, such as poly(aspartic acid) (“P(Asp)”) that is rendered hydrophobic by chemical conjugation of a hydrophobic drug, or is formed through the association of two oppositely charged polyions, such as found in polyion complex micelles.

Studies of the preparation of biodegradable polymeric micelles have focused on the utilization of PEG for the formation of the hydrophilic shell. (See, X. Zhang, X., et al., Inter. J. Pharm., 132:195-206, 1996; Yokoyama, M., et al., J. Control. Release, 55:219-229, 1998 and Allen, C., et al., J. Control. Release, 63:275-286, 2000).

Poly(N-vinyl-2-pyrrolidone) (“PVP”) is a water-soluble, bio-compatible, amphiphilic polymer having a highly polar lactam group surrounded by an apolar methylene group in the lipid backbone and a methine group in the ring. In comparison with PEG, PVP exhibits a diversity of interactions towards non-ionic and ionic cosolutes. (See, Molyneux, P., Proc. Int. Symp. Povidone, 1-19, 1983). Binding takes place most markedly with molecules having long alkyl chains or aromatic moieties. Similar to PEG, PVP can also increase the in vivo circulation time of colloidal carriers and peptides/proteins. (See, e.g., Kamada, H. et al., Biochem. Biophys. Res. Commun., 257:448-453, 1999; Torchilin, V. P., J. Microencapsulation, 15:1-19, 1998). Furthermore, it has been shown that nanoparticles containing diblock copolymers of poly(D,L-lactic acid) and PEG aggregate after freeze drying. (See, De Jaeghere, F. et al., Pharm. Res., 16:859-866, 1999). This problem can be circumvented by the use of a lyoprotectant, such as PVP.

N-vinyl pyrrolidone (“VP”) may be copolymerized with a wide variety of vinyl monomers. When VP is reacted with electronegative monomers, VP forms alternating copolymers. VP coupled with acrylates will yield random copolymers. For instance, a graft copolymer composed of poly(L-lactide) (“PLLA”) and PVP has been prepared. (See, Eguiburu, J. L., et al., J. San Roman, Polymer, 37:3615-3622, 1996).

Several studies additionally describe the use of poorly or non-biodegradable polymers such as polystyrene or poly(methyl methacrylate) (“PMMA”) as constituents of the inner core. (See, e.g., Zhao, C. L. et al., Langmuir, 6:514-516, 1990; Zhang, L. et al., Science, 268:1728-1731, 1995 and Inoue, T. et al., J. Controlled Release, 51:221-229, 1998). To be considered clinically relevant as drug carriers, non-biodegradable polymers need to be non-toxic and have a molecular weight sufficiently low to allow renal excretion. The hydrophobic inner core of such non-biodegradable polymers may be comprised of a highly hydrophobic small chain such as an alkyl chain or a diacyl lipid such as distearoyl phosphatidyl ethanolamine (“DSPE”). The hydrophobic chain may be attached to one end of a polymer or randomly distributed within the polymeric structure. The outer shell of the non-biodegradable polymer micelle is responsible for stabilization of the micelle and interaction with plasmatic proteins and cell membranes. Such micelles may consist of chains of hydrophilic, non-biodegradable, biocompatible polymers such as PEO. The biodistribution of such carriers is mainly dictated by the chemical identity of the hydrophilic shell. Polymers such as poly(N-isopropylacrylamide) (“PNIPA”) and poly(alkylacrylic acid) impart temperature or pH sensitivity to the micelles and may be used to confer bioadhesive properties. Micelles presenting functional groups at their surface for conjugation with a targeting moiety are also known. (See, e.g., Scholz, C. et al., Macromolecules, 28:7295-7297, 1995).

Methods for disrupting polymeric micelles include, for instance, application of thermal energy, application of ultrasound, or pH modification. (See, U.S. Pat. No. 5,955,509 and U.S. Pat. No. 6,649,702, the contents of which are incorporated by this reference.)

### DISCLOSURE OF INVENTION

Some inoperable cancerous tumors manifest a high resistance to systemic chemotherapy. In some cases, this is due to the action of drug efflux pumps encoded by multidrug resistant (“MDR”) or MRP genes. In other cases, drug resistance results from poor blood supply to the tumor volume. One method of treating such tumors is by direct injection of a chemotherapeutic agent. However, this method may present other obstacles, such as precise imaging of the tumor and efficient delivery of the chemotherapeutic agent to only the tumor mass.

The invention promises to overcome two main complications of cancer chemotherapy: severe side effects of toxic drugs and resistance of cancerous cells to drug action. A rationale behind the invention is that drug encapsulation in micelles or other nanoparticles decreases systemic concentration of free drug, diminishes intracellular drug uptake by normal cells, and provides for a passive drug targeting to tumor interstitium via the enhanced penetration and retention (EPR) effect, due to the abnormal permeability of tumor blood vessels (See, e.g., S. K. Hobbs et al., Proc. Natl. Acad. Sci. USA 95 (1998) 4607-4612). Drug targeting to tumors reduces unwanted drug interactions with healthy tissues (See, e.g., K. Kataoka et al., Adv. Drug Deliv. Rev. 47 (2001) 1130-1144; G. S. Kwon et al., Adv. Drug Deliv. Rev. 16 (1995) 295-309; K. Kataoka et al., J. Control. Rel. 64 (2000) 143-153). Upon micelle or nanoparticle accumulation in the tumor interstitium, an effective intracellular drug uptake locally by the tumor cells is triggered by tumor irradiation with therapeutic ultrasound (See, e.g., U.S. Pat. No. 6,649,702, the contents of which are herein incorporated by this reference). Ultrasonic irradiation results in an increased drug influx into tumor cells, which helps to overcome drug resistance caused by the work of the efflux pumps (See, e.g., Rapoport, N. (2003) Controlled Drug Delivery to Drug-Sensitive and Multidrug Resistant cells: Effects of Pluronic Micelles and Ultrasound. in: Advances in Controlled Drug Delivery. ACS Symposium Book Series, ed. S. Dinh and P. Liu, Washington D.C., 2003, pp. 85-101). For poorly vascularised tumors, direct drug injection through the ultrasound-guided syringe needles allows delivering drug to tumor, upon which tumor irradiation by therapeutic ultrasound enhances drug diffusion resulting in a more uniform drug delivery to tumor cells (See, e.g., Gao et al., J. Control. Release 102 (2005) 203-221).

Disclosed herein are, inter alia, methods and compositions for treating tumors by, for example, intravenous or direct intratumoral injection of microbubbles and polymeric micelles (formed in aqueous solutions of diblock or triblock copolymers or mixtures thereof) containing an agent, or agents, useful in treating tumors. Methods and compositions disclosed herein are especially useful in treating inoperable drug sensitive and/or drug resistant tumors, poorly vascularized tumors, and more especially inoperable poorly vascularized tumors. The disclosed intratumoral delivery methods are also beneficial for treating tumors with well-defined primary lesions, such as breast, colorectal, prostate, and skin cancers. As shown herein, a complete resolution of tumors may be obtained using the methods and compositions as disclosed herein. Additionally, these methods and compositions, as disclosed herein, are not limited to the aforementioned non-limiting examples, such as poorly vascularized tumors. These methods and compositions may be applied and easily adapted to broadly treat a myriad of different tumors, but especially may be useful in treating MDR tumors whose resistance may be caused, for instance, by the expression of MDR or MRP genes that encode drug efflux pumps.

A further embodiment of the present invention further includes, inter alia, use of a micelle disruption technology or technologies, such as ultrasound. Ultrasound may be applied extracorporeally, intraluminally, or interstitially. The mode of ultrasound application can be determined depending on the locations of the tumors. Ultrasound may be applied extracorporeally, as disclosed in the examples presented herein. HIFU (high intensity focused ultrasound) treatment (tumor ablation) uses extracorporeal ultrasound transducers. However, ultrasound can also be applied intraluminally via the endoscopic applicators, or interstitially via specially designed needle applicators. These applicators are in the market and are known in the art. Treating esophageal, pancreatic, or bile track tumors may be done with endoscopic applicators. Intravaginal applicator has already been used for treating uterine fibrosis.

Additionally, disclosed is a newly discovered class of polymeric drug carriers that simultaneously serve as ultrasound imaging contrast agents and enhancers of ultrasound-mediated micelle disruption. These nanoparticles, or microbubbles, formed in situ from a microemulsion composition with microdroplets stabilized by diblock, triblock copolymers, or mixtures thereof, may be used for the image-guided intratumoral treatment of tumors. These compositions, as disclosed herein, may be useful for many applications where images of tissues are desired.

The microbubbles can be composed of biocompatible gases or pharmacologically acceptable gases, for example, as described in U.S. Pat. No. 5,558,854 or U.S. Pat. No. 6,132,699, the contents of which are incorporated by this reference. The gas may comprise a single compound or a mixture of compounds. In general, many fluorine-containing gases are good candidates for forming microbubbles. In a preferred embodiment, microbubbles or nanobubbles are composed of perfluorocarbons (PFCs). Some PFCs, for example C₅F₁₂, are liquid at room temperature but convert into highly echogenic nano/microbubbles at higher temperatures, such as physiological temperatures. These substances may be called liquid/gas. Physiological temperature of various living organism may vary, for example, as disclosed in U.S. Pat. No. 5,558,854.

In various embodiments of the inventions, emulsion droplets or bubbles of various sizes can be formed. Various techniques in the art can be used to form microemulsions/microbubbles, for example, as described in U.S. Pat. No. 5,558,854. In a preferred embodiment of the invention, emulsion droplets are stabilized by polymeric copolymers. In a particular embodiment, emulsion droplets are stabilized by copolymers, and emulsion droplets/bubbles co-exist with polymeric micelles. In a more particular embodiment, a sterilized perfluorocarbon solution is mixed with a sterilized micellar solution, and forms nanoemulsions and microbubbles. Sterilization can be performed using various techniques known in the art, for example, irradiation or filtration.

In various embodiment of the invention, amphiphilic substances can be employed to form micelles and/or stabilize microbubbles. An amphiphilic molecule usually comprises a polar, water-soluble part and a nonpolar, water-insoluble part. Examples of amphiphilic substances include but are not limited to surfactants, detergents, lipids, certain proteins, certain polysaccharides, certain modified proteins or polysaccharides, certain polymers, and certain copolymers. In a preferred embodiment of the invention, block copolymers are used for formation of micelles and/or microbubbles. Examples of block copolymers include PEG-PLLA (poly(ethylene oxide)-block-poly(L-lactide)), PEG-PCL (poly(ethylene oxide)-block-poly(caprolactone)), and Pluronic P-105. The size and/or properties of the micelles and/or emulsion droplets can be controlled by factors such as copolymer type, block ratio, block length, copolymer concentration, and/or concentration of liquid/gas. For example, for the equivalent copolymer and PFC concentrations, emulsion droplet sizes are smaller for PEG2000-PCL2000 than PEG2000-PLLA2000 copolymer. Depending on these and/or more factors, different ratios (molar or volume ratios) of micelles and emulsion droplets may be obtained. Micelles may coexist with emulsion droplets. Under particular conditions, the system may contain only micelles, or only emulsion droplets. In a particular embodiment, essentially all block copolymers are used to stabilize emulsion droplets, and thus the concentration of free copolymers is below their corresponding critical micelle concentration (CMC) or mixed critical micelle concentration, therefore, no micelles are formed.

In various embodiments of the invention, the micelle/emulsion system may comprise other materials, such as a viscosity enhancer, e.g., water soluble polypeptides or carbohydrates and/or surfactants, to stabilize emulsion droplets. The system may also comprise pharmaceutically acceptable carriers, such as saline, glycerol, TWEEN™ 20, etc. U.S. Pat. No. 4,466,442 discloses various techniques for producing suspensions of gas microbubbles in a liquid carrier using a solution of a surfactant in a carrier liquid and a solution of a viscosity enhancer as stabilizer.

In various embodiments of the invention, block copolymers may comprise various polymer building blocks. Building blocks and formation of block copolymers are known in the art. Examples of polymer blocks include but are not limited to the representative synthetic polymers as described in U.S. Pat. No. 5,837,221.

In one embodiment of the invention, particle sizes change upon system heating to physiological temperatures. First, the droplets convert into microbubbles of larger sizes; at a longer incubation, the largest microbubbles gradually evaporate releasing a stabilizing copolymer that self-assembles into micelles.

In various embodiment of the invention, the shape of the micelle or emulsion bubbles may be of spherical or non-spherical shape. For example, a micelle can assume a shape of spherical, non spherical, cylindrical, or worm-like. The shape of a micelle may change depending on factors, such as co-surfactant, temperature and/or ionic strength of the system.

The direct intratumoral drug delivery methods disclosed herein have many advantages. For instance, such methods significantly reduce the side effects of commonly-used systemic chemotherapy treatments and therapeutic doses of drugs. Such side effects are commonly caused by activity of the chemotherapeutic drugs on non-target tissues, that is, non-tumorous or non-cancerous tissues. The precision achieved by delivering the chemotherapeutic agent, or agents, directly to only the target tissues thus may dramatically decrease common quality-of-life diminishing side effects associated with traditional chemotherapy methods.

Direct intratumoral drug injection requires precise positioning of the syringe needle in the tumor volume. This task is challenging, especially for tumors situated deep within tissues. The echogenic microbubbles disclosed herein address this problem because they combine the properties of drug carriers and ultrasound contrast agents into one convenient and efficient application. A stream of empty microbubbles (i.e. not drug-loaded microbubbles) may be used for the visualization of the syringe needle tip for needle guiding. The microbubbles described herein are produced in situ upon the injection of the specially designed microemulsion compositions as disclosed herein. As the microdroplet of the microemulsion compositions transforms into microbubbles, the echogenecity and acoustic cavitation properties are substantially increased. (See, E. Ungar et al., Advanced Drug Delivery Reviews, 56:1291-1314, 2004).

A further embodiment includes the disclosed microemulsion compositions that transform into microbubbles upon injection, in situ, for use in intravenous or intratumoral injection. Such microbubbles may be “loaded” with chemotherapeutic agents, for example, as disclosed herein. Upon injection, the area of the tumor or tissues may be exposed to energy, such as ultrasound, to break down the microbubbles and release the chemotherapeutic agent or agents. If injected intravenously, drug-loaded microbubbles could allow effective drug targeting to tumors because, as disclosed herein, the drug will be delivered predominantly to the sites that are locally irradiated by ultrasound. (See, Z. Gao, et al., J. Control. Release, 102:203-221 (2005); Z. Gao, et al., Molecular Pharmaceutics, 1:317-330 (2004); and N. Y. Rapoport, et al., Ultrasonics, 42:943-950 (2004)). Drug released from microbubbles locally in the tumor microvasculature may induce blockage or collapse of tumor capillaries thus blocking blood supply to tumors. In the MHz ultrasound range, localization of acoustic energy is possible in millimeter and even submillimeter volumes, allowing precise control of drug delivery. No microbubbles having the drug delivery properties of the microbubble compositions disclosed herein are currently commercially available.

In one embodiment of the agent-loaded micelle/emulsion droplets, loaded agent is partitioned between micelles and emulsion droplets or bubbles. Partitions of the drug between micelles and emulsion droplets/bubbles depend on the types of the materials that form the micelles and emulsions, and thus depend on the sizes or the ratios of the micelles and emulsion droplets. The agent may be enclosed inside of the micelles, or embedded in the polymer chains within the micelles, or located on the surface of the micelles. The drug molecules may also be enclosed inside of the emulsion droplets/bubbles, or embedded in the layers enclosing the droplets/bubbles, or located on the surface of the droplets/bubbles. It is possible that there are agents not included in or on the micelles or emulsion droplets, but dissolved or dispersed in the system outside of the micelles or emulsion droplets/bubbles.

In various embodiments of the invention, the micelles and emulsion droplets/bubbles can be designed for optimal delivery of a specific therapeutic agent. For example, using techniques known in the art, the types of the building blocks of a copolymer, the lengths of the blocks, the amount of copolymer used, the types of the liquid/gas, and/or the amount of liquid/gas, can be optimized for a therapeutic agent, depending on the size, charge, hydrophobicity, etc., of the therapeutic agent.

In various embodiment of the invention, a therapeutic agent can be loaded to the micelles, prior to generation of microemulsions. A therapeutic agent can also be included to the already mixed system of micelles and microemulsions. Various techniques of including therapeutic agent to colloidal systems have been developed in the field of drug formulation and delivery. It is to be noted that a therapeutic agent may be incorporated to a colloidal system via covalent or non-covalent linkages. In an exemplary embodiment, a therapeutic agent such as paclitaxel is physically entrapped in a mixed micelle/emulsion system. Upon formation of microbubbles, the high echogenecity of microbubbles guides delivery of paclitaxel to a tumor site, and/or application of ultrasound irradiation enhances uptake of the drug.

In another embodiment of the invention, the in situ-produced microbubbles may be used in many other applications, such as blood pool contrast agents, as enhancers of vascular thrombosis treatment, and as enhancers of gene delivery.

A further embodiment of the invention involves the use of the presently disclosed methods and drugs to treat inoperable, drug-sensitive, poorly vascularized, multidrug resistant, and well defined primary lesions such as breast, colorectal, prostate, and skin cancers.

Another embodiment of the invention includes application in diagnostic ultrasound imaging and gene delivery. The microbubbles formed in situ, upon injection, into the tissue, of the microemulsion, have been found to be detectable using ultrasound imagers, allowing imaging of tissues. Such imaging provides accuracy and specificity in guiding any treatment of any tissue with any agent or simply to provide an image of a tissue, such as a snapshot of the tissue structure prior to treatment, said treatment being surgical, chemical, or otherwise.

A keen interest exists in combining the drug carrier properties of micelles and ultrasound contrast agent properties of microbubbles into one composition. To date, no such compositions exist. The microbubble compositions currently commercially available are either albumin- or lipid-coated, stable gas bubbles, such as DEFINITY®, Bristol-Meyers Squibb and OPTISON®, Amersham Health, and do not have drug delivery properties and are quite costly.

The microbubble compositions disclosed herein have the following unique and advantageous properties (the following is not an exhaustive list and not intended to in any way to be limiting):
produced in situ upon injection of a specially designed microemulsion, the microemulsions being stable and allowing a long shelf life at room temperature and allowing freezing and thawing;
strong microbubble walls that are produced by a biodegradable diblock copolymer that stabilizes the microbubbles prior to ultrasonic imaging and/or application of therapeutic ultrasound;
effective encapsulation of chemotherapeutic agents by the same biodegradable diblock copolymer micelles, acting as drug carriers;
effective intracellular drug uptake by tumor cells, for both the released and micelle-encapsulated drug, upon injection and upon localized ultrasonic irradiation of the tumor (See, Z. Gao, et al., J. Control. Release, 102:203-221 (2005); Z. Gao, et al., Molecular Pharmaceutics, 1:317-330 (2004); N.Y. Rapoport, et al., Ultrasonics, 42:943-950 (2004); N. Rapoport, International Journal of Pharmaceutics, 277:155-162 (2004); Rapoport, N., Factors Affecting Ultrasound Interactions with Polymeric Micelles and Viable Cells, Carrier-Based Drug Delivery, American Chemical Society Symposium Series, S. Swenson, Ed., Washington, D.C., 879:161-173; U.S. Pat. No. 6,649,702; and Rapoport, N., et al., Drug Delivery Systems and Sciences, 2:37-46 (2002));
ability to effectively operate as a gene delivery agent; and
ability to enhance the ultrasonic treatment of vascular thrombosis.

For intratumoral injections, a long retention of the drug in the tumor volume is desired. As described herein, this can be achieved by drug encapsulation in the polymeric micelle/microbubble compositions disclosed herein. Drug encapsulation also prevents drug uptake by normal cells, thus reducing the side effects of chemotherapy.

As described herein, in another embodiment of the present invention, the efficient drug uptake by cancerous cells may be further enhanced by application of energy, such as in the form of ultrasound or sonication. Both nanoemulsion and nano/microbubbles are proved to be highly echogenic. As disclosed hereinbelow, a local ultrasonic irradiation of the tumor, after injection of the drug encapsulated by the micelle/microbubble compositions disclosed herein, triggers drug release from micelle composition within the tumor volume and enhances the intracellular drug uptake by the tumor cells.

### BRIEF DESCRIPTION OF DRAWINGS

FIG. 1. Schematic representation of one embodiment of the invention. Drug encapsulated in micelles and/or small microbubbles (diameter up to several hundred nanometers, depending on the tumor type) is extravasated in the tumor interstitium; drug encapsulated in large macrobubbles (diameter 700 nm or higher) remains in the circulation. These larger microbubbles serve as cavitation nuclei. Under the action of ultrasound directed to a tumor, the microbubbles oscillate and collapse thus triggering drug release from micelles and/or small nanobubbles in the tumor volume and enhancing the intracellular drug uptake by tumor cells.

FIG. 2. Microphotograph of a perfluoropentane (“PFP”) microemulsion in a micellar solution of poly(ethylene glycol)-co-poly(L-lactide) (PEG2000-PLLA2000).

FIG. 3. (A) Size distribution at room temperature of microemulsion of the composition of FIG. 2. (B) Size distribution at 42° C. of the microbubbles of the composition of FIG. 2. (C) Size distribution after cooling from 42° C. to room temperature of the microbubbles of the composition of FIG. 2.

FIG. 4. Effect of heating to 37° C. on the particle size distribution for PEG2000-PCL2000 1%, PFP 1% system. Upon heating, nanodroplets (regular line) convert into larger microbubbles (bold line); for PEG-PCL, the heating effect is reversible: upon cooling, the initial particle size distribution is effectively restored (dotted line).

FIG. 5. Particle size distribution for PEG2000-block-PLLA2000 copolymer (1.0%) at PFP concentrations of 0.1% (v/v). (A) and 1% (v/v) (B). (A) shows predominantly micelles (21.4 nm); (B) shows predominantly droplets (256 and 811 nm). Particle sizes shown herein is the size corresponding to the peak maximum.

FIG. 6. Particle size distribution for a PEG2000-block-PLLA2000 concentration of 0.2% and a PFP concentration of 0.5%. Compare a droplet size of 1800 nm at a copolymer concentration of 0.2% to 800-1200 nm at a copolymer concentration of 1.0%.

FIG. 7. Mean bubble sizes in PEG2000-PLLA2000 0.5%-PFP 2%-DOX 0.75 mg/ml system incubated for various times at 37° C. Bubble size was measured at room temperature (RT). This system was further used in cell culture and animal experiments. The data shown above imply that the nano/microbubble will be preserved in the circulation for at least 4 h upon intravenous injections. The particle size distribution after 4 h of heating is shown in FIG. 19 for two independent trials.

FIG. 8. Particle size distribution after 4 h heating of the PEG2000-PLLA2000 0.5%, PFP 2%, DOX 0.75 mg/ml formulation at 37° C. Results of two independent experiments are shown. Nanobubbles of 700 nm-800 mm size and smaller nanodroplets of 200 nm-300 nm are preserved after a 4 h heating. Smaller nanodroplets are expected to penetrate through tumor capillary walls and accumulate in the tumor interstitium. Larger bubbles will remain in circulation; their collapse under tumor-localized ultrasound enhances drug release from smaller nanoparticles and the intracellular drug uptake by the tumor cells.

FIG. 9. Dependence of particle size distribution on the PFP concentration for Pluronic P-105 concentration of 1.0%. Micelles were observed at a PFP concentration of 0.1% but not at a PFP concentration of 0.5% or 1% when only nanodroplets (580 nm-680 nm) exist in the system. Size distribution was measured one day after emulsion preparation and the system was kept at RT.

FIG. 10. Particle size distribution in the initial PEG2000-PCL2000 solution (no PFP).

FIG. 11. Particle size distribution in the PEG2000-PCL2000 0.3%-PFP 1.0% system.

FIG. 12. Particle size distribution in the PEG2000-PCL2000 0.3%-PFP 1.0% system diluted 45-fold.

FIG. 13. Effect of PEG2000-PCL2000 copolymer concentration on the nanobubble size (PFP concentration 1%).

FIG. 14. (A) Particle size distribution before sonication. (B) Particle size distribution after sonication by focused 1.1-MHz ultrasound at RT (ultrasound parameters: frequency 1.1 MHz, pressure amplitude 0.55 MPa, duty cycle 30%, duration 15 s, pulse length 0.5 s). Large droplets (1500 nm) are broken into smaller droplets (657 nm). The number of the droplets increases leading to a disappearance of micelles. Ultrasound does not have effect on smaller (274 nm) droplets. (C) Particle size distribution after sonication by unfocused 1-MHz ultrasound at 37° C. Ultrasound parameters: 1.0 MHz, power density 3.4 W/cm2, duty cycle 33%, duration 30 s. The effect is similar to that observed at RT (See, FIG. 10 c) except that more droplets are collapsed to shift the distribution in favor of small copolymer associates (130 nm). (D) Particle size distribution after sonication by continuous wave (CW) unfocused 1-MHz ultrasound at RT. Ultrasound parameters: CW, 1.0 MHz, power density 3.4 W/cm2, duration 60 s. The microdroplets convert into microbubbles and undergo collapse. The released copolymer molecules self-assemble into micelles (28 nm). (E) Particle size distribution after sonication of a PEG2000-PLLA2000 0.5%, PFP 2% system by unfocused 3.0-MHz ultrasound at 37° C. Ultrasound parameters: 3.0 MHz, power density 2.0 W/cm2, duty cycle 20%, exposure duration 30 s. Droplets are collapsed; the population of micelles (28 nm) is formed.

FIG. 15. Phase contrast (left) and fluorescence (right) micrographs of PEG2000-PCL2000 DOX-loaded microbubbles.

FIG. 16. Enhancing effect of ultrasound on the DOX delivery to ovarian carcinoma A2780 cells from PEG2000-PLLA2000 micelles in the absence of microbubbles. A significant enhancement of the intracellular drug uptake is observed. The dashed line represents the control sample (untreated A2780 cells). The light solid line represents treatment of A2780 tumor cells with micelle encapsulated DOX, no ultrasound applied. The dotted line represents A2780 tumor cells treated with micelle encapsulated DOX, ultrasound was applied at 3 MHz. The solid dark line represents A2780 tumor cells treated with micelle encapsulated DOX, ultrasound was applied at 1 MHz.

FIG. 17. Effect of microbubbles on the DOX delivery to ovarian carcinoma A2780 cells using the PEG2000-PLLA2000 micelle/microbubble system. The presence of microbubbles in sonicated samples additionally enhances the intracellular uptake of DOX. The dashed line represents control cells. The light solid line represents A2780 tumor cells treated with micelle encapsulated DOX, ultrasound was applied at 3 MHz. The dark solid line represents A2780 tumor cells treated with micelle encapsulated DOX, ultrasound was applied at 3 MHz, and microbubbles were co-administered.

FIG. 18. Effect of microbubbles and ultrasound on the intracellular DOX uptake by the cells of the excised MDA MB 231 tumor. (A) No microbubble. (B) Microbubbles. Formulation: [DOX]=0.75 mg/ml; [PEG-PLLA]=0.5%; [PFP]=2%. Ultrasound parameters: unfocused ultrasound, frequency 3 MHz, power density 2 W/cm², duty cycle 50%, duration 1 min. Measurement is based on the intracellular DOX fluorescence.

FIG. 19. Effect of microbubbles and ultrasound on the intracellular DOX uptake by the multidrug resistant ovarian carcinoma A2780/AD cells; DOX (20 μg/ml) was delivered in PEG-PLLA micelles. Ultrasound parameters: unfocused ultrasound, frequency 1 MHz, power density 3.4 W/cm², duty cycle 33%, duration 1 min.

FIG. 20. Florescence histograms of the multidrug resistant ovarian carcinoma A2780/AD cells after 0.5 second sonication by 1.1 MHz focused ultrasound (33% duty cycle, 0.5 second pulse, 5 MPa) in the presence of (A)—microemulsion at 25° C. and (B)—microbubbles at 37° C.

FIG. 21. Viability of multidrug resistant ovarian carcinoma A2780/AD cells after 0.5 second sonication by 1.1 MHz ultrasound (33% duty cycle, 0.5 second pulse length, 5 MPa) in the presence of the microemulsion at either 25° C. or 37° C. Microbubbles dramatically enhance the ultrasound-induced reduction of the number of viable multidrug resistant ovarian carcinoma cells.

FIG. 22. Photograph of a nu/nu mouse with A2780 human ovarian cancer tumor exposed.

FIG. 23. Growth curves of A2780 ovarian carcinoma tumors inoculated in female nu/nu mice. Intravenous treatments from top to bottom: untreated control; DOX dissolved in PBS (free DOX); DOX encapsulated in mixed PLURONIC® P-105 (BASF)/PEG2000-DSME micelles; DOX encapsulated in mixed PLURONIC® P-105/PEG2000-DSME micelles and treated by unfocused 1 MHz ultrasound for 30 seconds at 3.4 W/cm² and 50% duty cycle. DOX was administered at 3 mg/kg and ultrasound was applied 4 hours after intravenous injection of DOX.

FIG. 24. The interior of a poorly vascularized HCT116 human colon cancer tumor in male nu/nu mice.

FIG. 25. HCT116 human colon cancer tumor bearing male nu/nu mouse before intratumoral treatment with polymeric micelle-encapsulated DOX.

FIG. 26. HCT116 human colon cancer tumor bearing male nu/nu mouse after intratumoral treatment with polymeric micelle-encapsulated DOX.

FIG. 27. Growth curves of HCT116 tumors upon intravenous and intratumoral injection of doxorubicin (“DOX,” ADRIAMYCIN® or RUBEX®). Solid diamonds represent control samples. Open squares represent tumors treated with DOX dissolved in phosphate-buffered saline (“PBS”) administered intravenously. Open triangles represent micelle encapsulated DOX intravenous treatment followed by ultrasound. Open circles represent micelle encapsulated DOX intratumoral treatment. Closed circles represent micelle encapsulated DOX intratumoral treatment followed by ultrasound. Dramatic differences in the effects of the intravenous versus intratumoral injections are observed on the growth of HCT116 colon cancer tumors.

FIG. 28. Fluorescence histograms of the tumor cells ten hours after the intratumoral injections of free or micelle-encapsulated DOX: the thin line represents the control (HCT116 tumor cells in untreated mice), the dotted line represents DOX solubilized in PBS (free DOX), the thick line represents micelle-encapsulated DOX.

FIG. 29. The photographs of the mice inoculated with breast cancer MDA MB231 tumors. Mice were treated by 3 mg/kg DOX encapsulated in PEG2000-PLLA2000 (0.5% polymer in a PBS solution) formulated with 2% (v/v) PFP (denoted herein as a microbubble formulation). Some mice were sonicated for 150 s by unfocused 3-MHz ultrasound at 2 W/cm² power density and 20% duty cycle. Control tumors manifested dramatic growth (compare A and B); for the intravenous injections, treatment started when tumor reached a size presented in Panel C; tumor growth was completely inhibited (compare Panels C and D). For the intratumoral injections, treatment started when tumors reached sizes presented in Panels E and G; the treatment resulted in disease stabilization (compare F and E or H and G). Panel codes for FIG. 29 are included in Table 12.

FIG. 30. The photographs of a mouse inoculated with two multidrug resistant breast cancer MCF7/AD tumors (left panel). The left tumor was treated by four intratumoral injections (two times weekly) of a paclitaxel encapsulated in PEG-PLLA micelles; the treated tumor was sonicated for 30 s by unfocused 1-MHz ultrasound at 3.4 W/cm² power density and 33% duty cycle 15 min after the drug injection. The volume of the treated tumor gradually decreased (middle panel; photograph taken three weeks after the end of the treatment); a complete resolution of the treated tumor was observed a month and a half after the treatment (right panel).

FIG. 31. Ultrasound images at 7.5 MHz of phantom capillaries filled with (left to right): microbubbles at 37° C., microemulsion at RT, and water.

FIG. 32. Ultrasound images at 7.5 MHz of a stream of nanodroplets or microbubbles emanating from the syringe needle. (A) A syringe needle injecting a stream of a microemulsion composition, as disclosed herein, into beef liver at room temperature. (B) A syringe needle injecting a stream of a microbubble composition into an agarose gel, at 42° C. The stream of microbubbles emanating from the syringe needle tip and a microbubble cloud above the needle are visible.

### MODES FOR CARRYING OUT THE INVENTION

With the rapid advancement of live whole-organ tissue imaging techniques, an exact positioning of the injection needle in the tumor mass to inject chemotherapeutic agent is possible. In one aspect, the invention provides a viable solution for treating tumors, such as drug-sensitive tumors, inoperable tumors, poorly vascularized tumors, and MDR tumors. The direct intratumoral drug delivery to poorly vascularized tumors is a viable alternative to inefficient intravenous injections of the antineoplastic compositions. The injection needle or catheter may be inserted into the tumor under the ultrasound guidance followed by tumor irradiation by therapeutic ultrasound via either the inserted endoscopic ultrasonic transducer or by extracorporeal probe.

“Treating” or “treatment” according to the present invention does not require a complete cure. It means that the symptoms (such as, for instance, presence of tumors) of the underlying disease are at least reduced, and/or that one or more of the underlying cellular, physiological, or biochemical causes or mechanisms causing the symptoms are reduced and/or eliminated. It is understood that reduced, as used in this context, means relative to the state of the disease, including the molecular state of the disease, not just the physiological state of the disease.

Drugs, or agents, that may be used in the context of the present invention include those useful in treating tumors and cancers, antineoplastics, such as DOX, adriamycin, cisplatin, taxol, and 5-fluorouracil. Furthermore, other agents useful in the treatment of tumors, such as betulinic acid, amphotericin B, diazepam, nystatin, propofol, testosterone, estrogen, prednisolone, prednisone, 2,3 mercaptopropanol, and progesterone, may be co-administered with the polymeric micelles.

“Agent” as used herein can mean a biologically active agent, a drug, a pharmaceutical composition, an inert substance, an organic compound, an inorganic compound, a chemotherapeutic, a statin, an antineoplastic, or any combination of the preceding chemicals.

PEG2000-PLLA2000 is a copolymer with the molecular weights of the blocks of 2000 Da. PEG2000-PLLA5000 is a copolymer with the molecular weight of the poly(ethylene oxide block) of 2000 Da and that of poly(L-lactide block) of 5000 Da. Similar designations hold for other copolymers such as PEG-PCL.

“Droplet size” or “particle size” as used herein means the size (diameter) of the droplet or particle either at the maximum of the corresponding size distribution peak or as mean (volume-average) peak value, both measured by dynamic light scattering.

“Nanoparticle” as used herein refers to any object with a feature size, for example, diameter, smaller than or about one micrometer. Examples of nanoparticles include but are not limited to micelles, liposomes, bubbles and droplets. Typically, micelles have a size of 10-100 nm, liposomes have a size of 100-200 nm and nanoemulsions have a droplet size of 100-1000 nm.

“Room temperature” as used herein means an ambient temperature of about 18 to about 25° C.

“Microemulsions” as used herein also include nanoemulsions. “Microbubbles” as used herein also include nanobubbles. “Microdroplets” as used herein also include nanodroplets. As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.”

Dosages used of the respective drug or drugs (“biologically active agents” and antineoplastics) will depend on such variables as the particular drug used, the size and state of the tumor being treated, the frequency of administration, and the health of the patient.

The compositions disclosed herein may be formulated as pharmaceutically acceptable compounds or compositions. Excipients, diluents and/or carriers are known in the art, for example, See Remington's Pharmaceutical Sciences, 18th Ed. (1990, Mack Publishing Co., Easton, Pa.).

The present invention is further described in the following non-limiting examples, which are offered by way of illustration and are not intended to limit the invention in any manner.

### EXAMPLES

### Example 1

### Schematic Representation of Drug Targeting to Tumors Using Micelle/microbubble Systems Disclosed Herein

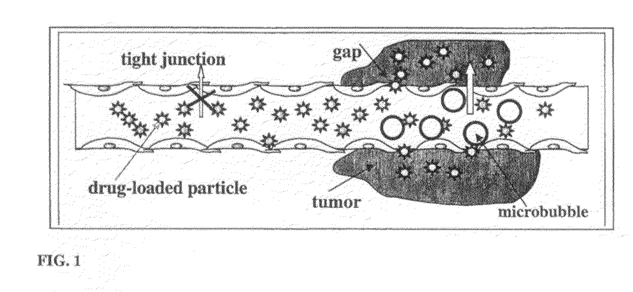

Drug targeting is based on the abnormal permeability of the tumor blood vessels due to relatively large gaps between endothelial cells (See, FIG. 1). Micelles and probably also small nanobubbles (up to 500 nm diameter, depending on the tumor type) extravasate and accumulate in the tumor interstitium. Larger nano/microbubbles remain in the circulation. Under tumor-localized therapeutic ultrasound, they serve as cavitation nuclei, triggering drug release from micelles and nanobubbles in the tumor interstitium and enhancing the intracellular drug uptake by the tumor cells.

### Example 2

### Synthesis of PEG/PLLA Diblock Copolymers

A library of amphiphilic PEG/PLLA diblock copolymers were synthesized by ring opening polymerization of L-lactide, initiated using the hydroxyl group of PEG monoacid in the presence of stannous octanoate as a catalyst. (See, Yamamoto, Y. et al., J. Contr. Release, 77:27-38 (2001); Kim, S C, et al., Particulate Drug Delivery, Samyang Pharmaceuticals R&D, Taejeon, 305-348, 2005, South Korea).

By varying the ratio of L-lactide to PEG, and the molecular weight of PEG, the PLLA block length and PEG-PLLA block length ratios are varied. The copolymers with the following block length were synthesized:

PEG: 2000 Da, 5000 Da

PLLA: 1000 Da, 2000 Da, 3000 Da, 5000 Da

### Example 3

### Characterization of PLLA/PEG Diblock Polymer by NMR and GPC

Reaction products are analyzed by ¹H-NMR (300 MHz). The Mₙ (median) and molecular weight distribution (Mw/Mₙ) of the synthesized copolymers are measured by gel permeation chromatography.

### Example 4

### Preparation of Dox-Loaded Micelles and Microemulsions

Micelles: Doxorubicin.HCl (“DOX.HCl”) is converted into the DOX base by incubation with triethylamine in dimethylsulfoxide (“DMSO”). DOX-loaded PEG2000-PLLA2000 copolymer micelles were produced using a solvent exchange technique. The joint solution in DMSO of copolymer and DOX base was dialyzed against PBS using a 2 kDa cut-off dialysis membrane (SpectraPor, Spectrum Medical Industries, CA, US). The amount of entrapped DOX was determined by HPLC. About 90-95% of the introduced DOX was encapsulated.

Microemulsions: Aliquots of perfluoropentane FLUORINERT™ Fluid PFP-5050 (3M™, St. Paul, Minn.) were added to DOX-loaded PEG2000-PLLA2000 or PEG2000-PCL2000 micelles. The micelles were then exposed to 20 kHz ultrasound for 15-30 second (Sonics & Materials, Inc., Newton, Conn.) to prepare DOX-loaded microemulsions formed in micellar solutions of PEG/PLLA copolymers. PFP concentration in PEG-PLLA micellar solutions may be varied in the range of 0.1% to 5% (v/v) to obtain varying droplet sizes and microbubble sizes. This is necessary for optimizing the echogenic properties for various applications, for instance, syringe needle positioning, blood pool imaging, and acoustic activation of drug delivery.

### Example 5

### Microbubbles Photomicrography

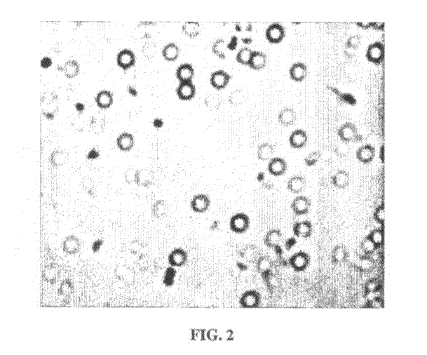

Microphotographs of microemulsions are presented in FIG. 2. As can be seen in FIG. 2, a narrow microemulsion particle size distribution is observed.

### Example 6

### Quantification of the Effect of Heating on the Particle Size Distribution in Micelle/Microemulsion System

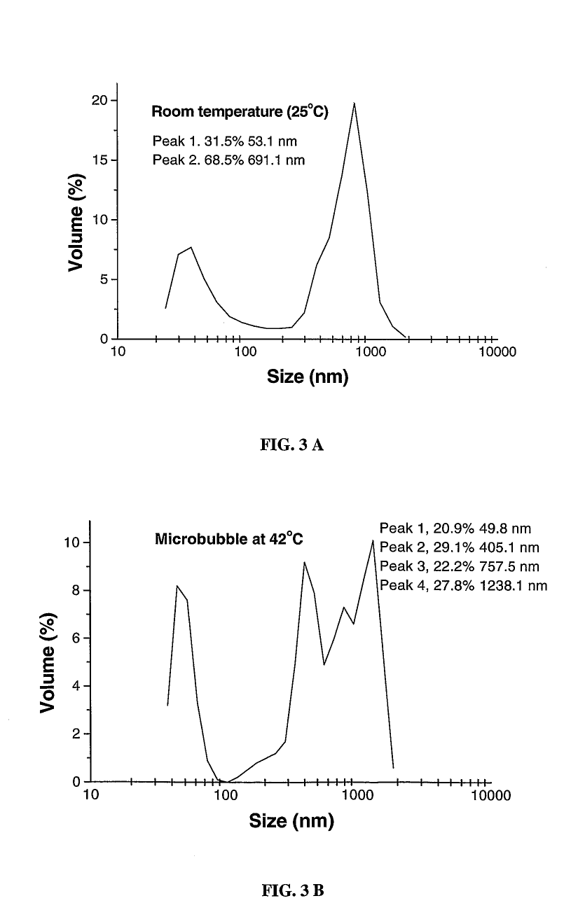

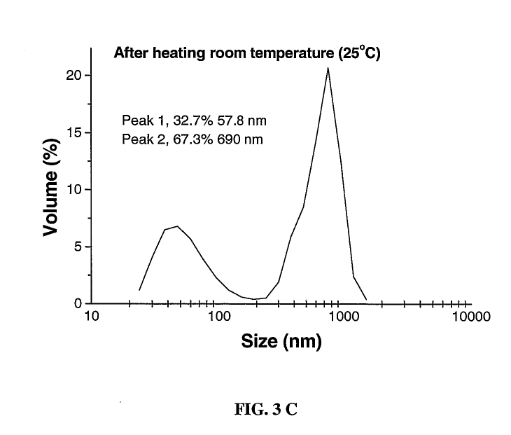

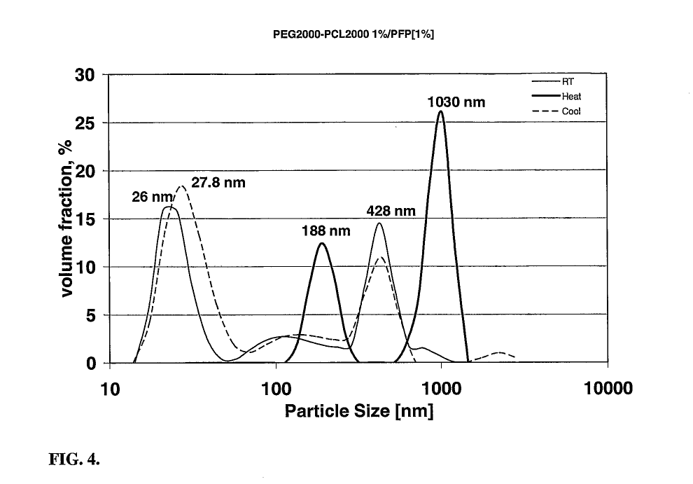

Particle sizes were measured using the Zetasizer3000 (Malvern Instruments, US). For the PEG2000-PLLA2000 copolymer, the particle size distribution manifested two peaks. (See, FIG. 3). At room temperature, the left peak of FIG. 3A (53.1 nm n) corresponds to PEG2000-PLLA2000 micelles. The right peak of FIG. 3A (691.1 nm) corresponds to the microemulsion droplets. Upon heating to 42° C., the perfluorocarbon droplets of the microemulsion boil to form microbubbles with a size distribution of between 750 nm and 1250 nm. (See, FIG. 3B). The process is reversible. Upon cooling to room temperature, the microbubbles condensed to produce a microemulsion of the initial particle size distribution. (See, FIG. 3C). Similar effects are observed for the nanodroplets stabilized by PEG2000-PCL2000 copolymer (See, FIG. 4).

### Example 7

Poly(ethylene oxide)-block-poly(L-lactide), PEG2000-PLLA2000 Stabilized Nanodroplets

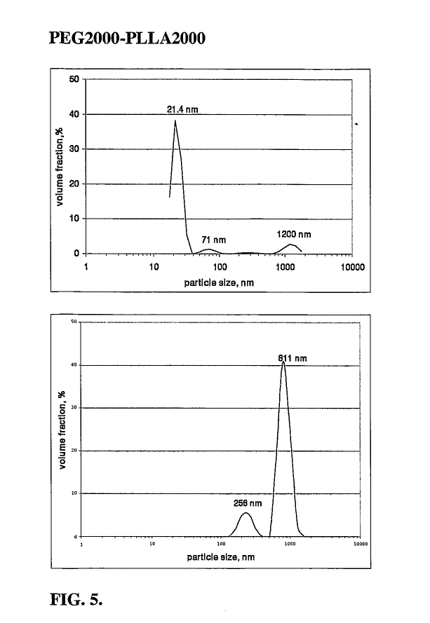

Phase state of the drug delivery system and droplet sizes may be controlled by the block length ratio of the copolymer, copolymer concentration, PFP concentration, and the copolymer/PFP concentration ratio. As shown in FIGS. 5-6, for 1% copolymer concentration, micelles and nano/microdroplets coexist in the system at PFP concentrations of 0.1% and 0.5%. However, no micelles were formed at PFP concentrations of 1% or 5%, because all copolymer molecules were used for the droplets stabilization, which stabilization process appears energetically more advantageous than copolymer molecules self-assembling in micelles. Bimodal droplet distribution was recorded for 1% PFP (256 nm and 811 nm, droplet sizes at the maximum of the corresponding peaks are presented). Smaller droplets might extravasate while larger ones will remain in the circulation (See, FIG. 1). Only one size of 595 nm was recorded for 5% PFP, which may be caused by the limit of the instrument sensitivity for the droplets larger than 3000 nm.

Droplet size may be controlled by copolymer concentrations. For the same PFP concentration, lower copolymer concentrations result in larger droplets (Tables 1 and 2). Effect of copolymer concentration on droplet size is also summarized in Tables 3 and 4.

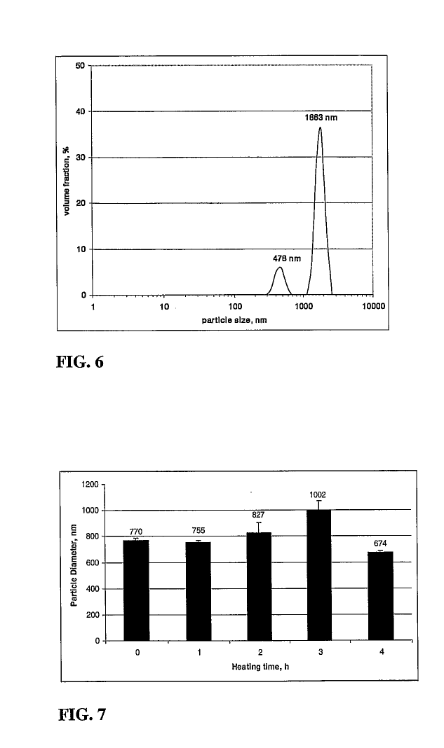

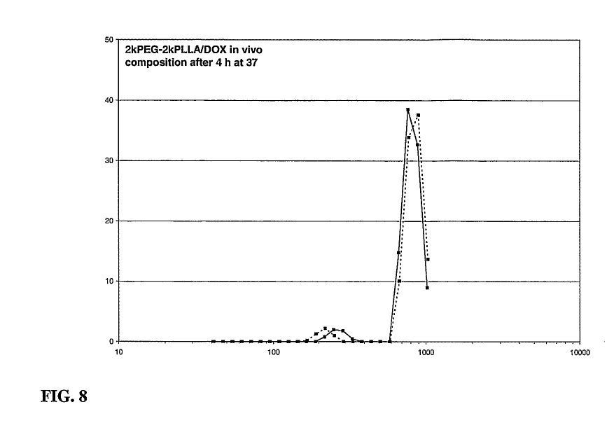

Micelles (24.1 nm) appear after heating-cooling cycles for the above systems due to the evaporation of some larger bubbles and release of the stabilizing copolymer, which then self-assembles into micelles; however a significant fraction of the droplets (762 nm and 1916 nm) still persist after the heating-cooling cycle (See, FIGS. 7 and 8). At a short-term heating (about a 5 min heating from RT to 42° C. followed by 5-15 min incubation at 42° C.) nanodroplets convert into nano/microbubbles; upon cooling to room temperature, the effect is completely reversible.

TABLE 1
Effect of PEG2000-PLLA2000 copolymer
concentration on droplet sizes.
PFP concentration 1.0%

| | | Particle |
|---|---|---|
| Copolymer | Particle size, | Volume |
| concentration | nm | Fraction, % |
| 0.2% | 445 | 43.5 |
| | 2207 | 56.5 |
| 1.0% | 256 | 13.6 |
| | 811 | 86.4 |

TABLE 2
Effect of PEG2000-PLLA2000 copolymer
concentration on droplet size.
(micelles are shown in bold fonts)
PFP concentration 0.1%

| | | Particle |
|---|---|---|
| Copolymer | Particle size, | Volume |
| concentration | nm | Fraction, % |
| 0.2% | 1509 | 100 |
| 0.5% | 264 | 13.6 |
| | 1319 | 86.4 |
| 1.0% | 22.8 | 87 |
| | 70.2 | 3.7 |
| | 277 | 1.2 |
| | 1236 | 8.1 |

Note that the number fraction of particles is proportional to the volume fraction divided by D³, where D is a particle diameter. Therefore a high volume fraction of large droplets may correspond to a small number of the droplets in the formulation. As well, presence of large droplets in the formulation may “mask” the presence of micelles. Therefore the presence or absence of micelles should be verified by the number fraction values.

For the same copolymer concentration, the size and especially the number of droplets increase dramatically with increasing PFP concentration (Tables 3 and 4).

TABLE 3
Droplet volume fraction increases with PFP concentration.

| Copolymer concentration 1% | | Copolymer Concentration 0.2% | |
|---|---|---|---|
| PFP | Droplet Volume | PFP | Droplet Volume |
| Concentration | Fraction | Concentration | Fraction |
| 0.1% | 9.3 | 0.1% | 100 |
| 0.5% | 11.6 | 0.5% | 100 |
| 1.0% | 100 | 1.0% | 100 |

For the PEG2000-PLLA2000 copolymer, micelles and droplets coexist in a narrow range of copolymer/PFP concentrations and only at a copolymer concentration of 1.0% (Table 4). At a copolymer concentration of 0.5%, nanoparticles of 100 nm to 260 nm coexist with larger droplets; the smaller particles are most probably small nanodroplets or the micelles with the PFP dissolved in the micelle core.

### Example 8

Poly(ethylene oxide)-block-poly(L-lactide), PEG2000-PLLA5000, Stabilized Nanodroplets

PEG2000-PLLA5000 has a longer hydrophobic block than PEG2000-PLLA2000. A strong interaction in the micelle core of PEG2000-PLLA5000 copolymer inhibits unimer diffusion out of micelles that is required for micelle/droplet equilibration and droplet stabilization. At a copolymer concentration of 1%, a very small number of droplets are formed even at a PFP concentration of 1% (compare 3% droplets for PEG2000-PLLA5000 to 100% droplets for PEG2000-PLLA2000). At any copolymer concentration, no droplets are formed for a PFP concentration of 0.1%. At a copolymer concentration of 1% and a PFP concentration of 5%, the droplets of 744 nm are formed. A decrease in a copolymer concentration allows droplet formation presumably because it results in a more hydrated and less strong micelle cores, which allows unimer diffusion out of micelles. The data for a PFP concentration of 1% is presented in Table 5.

TABLE 4
PEG2000-PLLA2000 copolymer and PFP concentration range for
micelles/droplets coexistence

| | | Micelle/ | |
|---|---|---|---|
| Copolymer | PFP | Microemulsion | |
| Concentration | Concentration | Coexistence | Particle Type and Size |
| 0.2% | 0.1% | No | Droplets 1509 nm |
| | 0.5% | No | Droplets |
| | | | 454 nm/1750 nm |
| | 1.0% | No | Droplets |
| | | | 451 nm/2147 nm |
| 0.5% | 0.1% | No* | Droplets 110 nm/440 |
| | | | nm |
| | 0.5% | No | Droplets 170 nm/540 |
| | | | nm |
| | 1.0% | No | Droplets 250 nm/ |
| | | | 1328 nm |
| 1.0% | 0.1% | Yes | Micelles/Droplets |
| | | | 22.8 nm/1236 nm |
| | 0.5% | Yes | Micelles/Droplets |
| | | | 24.0 nm/400-900 nm |
| | 1.0% | No | Nanoparticles/Droplets |
| | | | 226 nm/834 nm |

*Nanoparticles of 110 nm are probably large micelles with the PFP dissolved in the micelle core (rather than small nanodroplets).

TABLE 5
Effect of a PEG2000-PLLA5000 copolymer concentration
on droplet sizes; PFP concentration 1%.

| | | Particle |
|---|---|---|
| Copolymer | Particle size, | Volume |
| concentration | nm | Fraction, % |
| 0.2% | 264 | 10.2 |
| | 847 | 89.8 |
| 0.5% | 227 | 1.4 |
| | 768 | 98.6 |
| 1.0% | 17.0* | 97 |
| | 645 | 3 |

*Micelles are shown in bold fonts

Note that at a copolymer concentration of 0.2% and PFP concentration of 1%, the size of nanoparticles was larger for PEG2000-PLLA2000 than for PEG2000-PLLA5000 (2200 nm vs. 850 nm). Thus, the size of nanodroplets may be controlled by the length of the copolymer's hydrophobic block.

### Example 9

Poly(ethylene oxide)-block-poly(caprolactone), PEG2000-PCL2000 Stabilized Nanodroplets

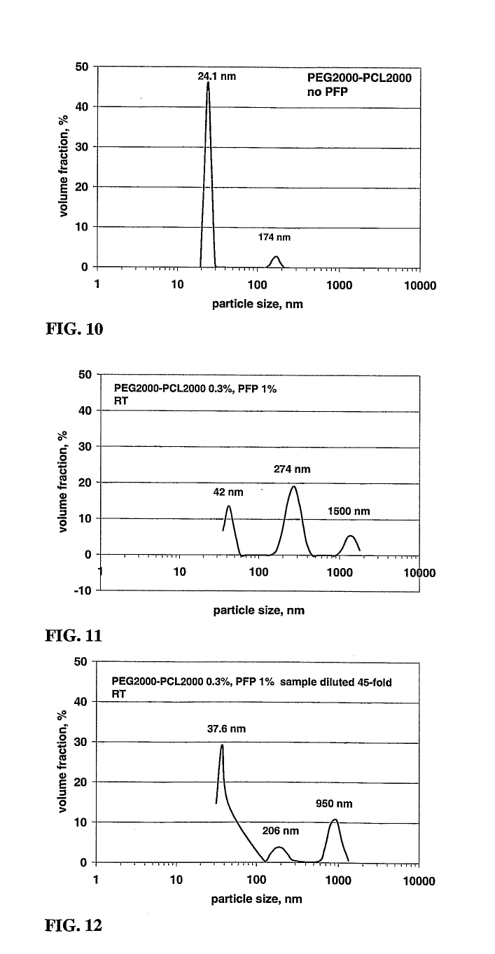

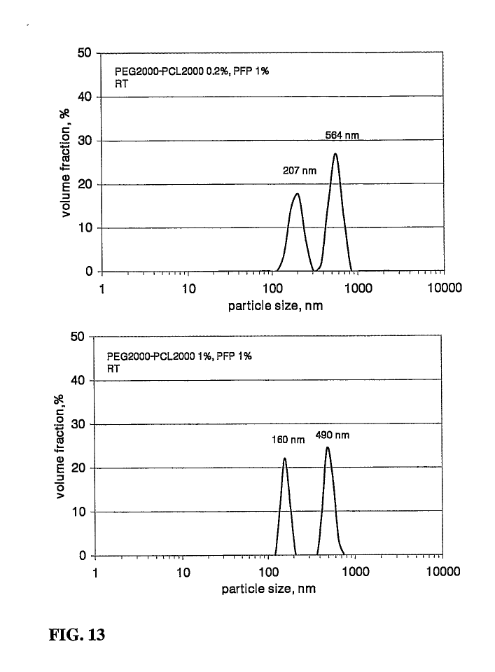

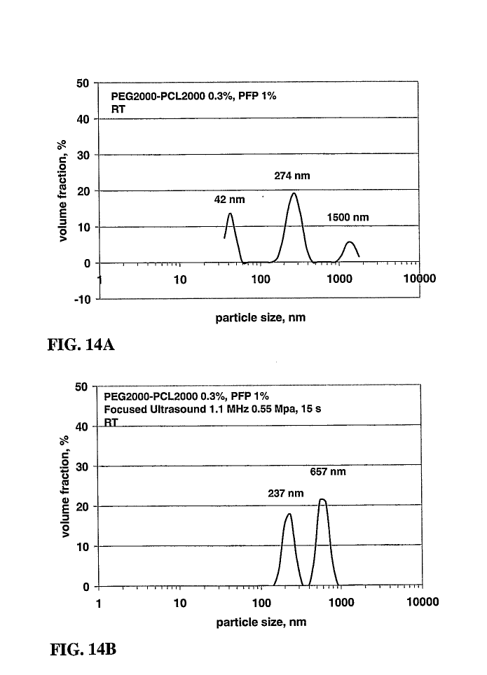

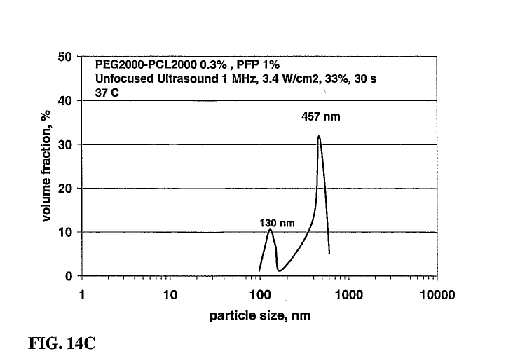

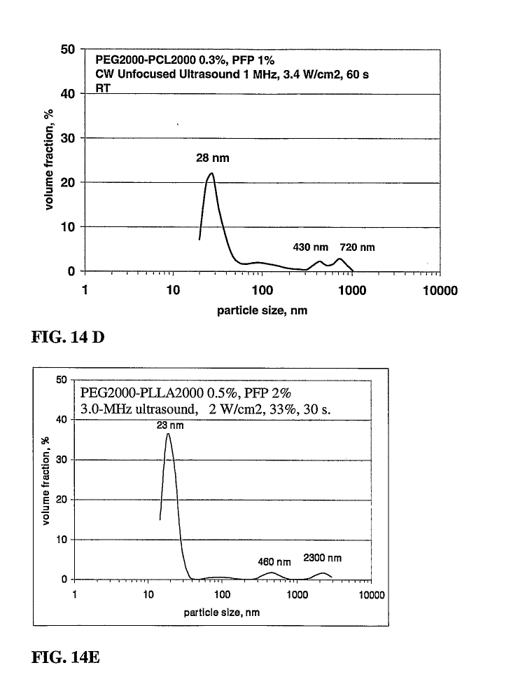

One important difference between the PEG2000-PLLA2000 and PEG2000-PCL2000 copolymers is a broader micelle/droplet coexistence range for the PEG2000-PCL2000 (Table 6) as compared to PEG2000-PLLA2000 (Table 4). Even at a low PEG-PCL copolymer concentration of 0.2%, micelles are preserved and coexist with nanodroplets for PFP concentrations of 0.1 and 0.5%. This finding makes PEG-PCL copolymers attractive candidates for fabrication of mixed micelle/microbubble formulations. More supporting data for this example can be found in FIGS. 10-14. Nanodroplets are preserved upon a significant system dilution (See, FIG. 11-12). Nanobubbles produced from nanodroplets are ultrasound-responsive (See, FIG. 14).

TABLE 6
PEG2000-PCL2000 copolymer and PFP concentration ranges
corresponding to the micelles and nano/microdroplet coexistence

| | | Micelle/ | |
|---|---|---|---|
| Copolymer | PFP | Microemulsion | Particle Type/Size |
| Concentration | Concentration | Coexistence | (nm) |
| 0.2% | 0.1% | Yes | Micelles/Droplets |
| | | | 53; 255 |
| | 0.5% | Yes | Micelles/Droplets |
| | | | 51; 225; 488 |
| | 1.0% | No | Droplets |
| | | | 207; 564 |
| 1.0% | 0.1% | Yes | Micelles/Droplets |
| | | | 21; 91; 417 |
| | 0.5% | Yes | Micelles/Droplets |
| | | | 29; 129; 479, 2000 |
| | 1.0% | No | Nanoparticles/ |
| | | | Droplets 160; 490 |

At corresponding PFP and copolymer concentrations, smaller nanodroplets are formed for PEG2000-PCL2000 as compared with PEG2000-PLLA2000 copolymer. The data for PEG-PCL are presented in Table 7. Compare droplet size of 2200 nm for PEG-PLLA to 580 nm for PEG-PCL (copolymer concentration 0.2%, PFP concentration 1%), the same trend is observed for copolymer concentrations of 1% (compare data of Tables 1 and 7).

Of a paramount importance is a higher stability of submicron (200 nm to 500 nm) PEG-PCL nanobubbles upon heating. While the bubbles of the same sizes stabilized by PEG-PLLA gradually evaporate and disappear upon incubation at 37° C., those stabilized by PEG-PCL are better preserved at the heating/cooling cycles. As was reported above for PEG-PLLA, larger microbubbles (micron size range) gradually evaporate, as exemplified in Table 8. When bubbles evaporate, the stabilizing copolymer is released; upon release, the copolymer molecules self-assemble in micelles, as manifested by a 1% copolymer/1% PFP formulation. Before heating/cooling, no micelles are observed in this formulation because all copolymer molecules are completely used for the nano/microbubble stabilization. Upon heating, the micron-size microbubbles evaporate while 24 nm micelles are formed.

TABLE 7
Effect of PEG2000-PCL2000 copolymer concentration on droplet sizes
(PFP concentration at 1%)

| | | Particle |
|---|---|---|
| Copolymer | Particle size, | Volume |
| concentration | nm | Fraction, % |
| 0.2% | 196 | 43 |
| | 578 | 57 |
| 1.0% | 161 | 44 |
| | 511 | 56 |

TABLE 8
Changes of the PEG2000-PCL2000 nanoparticle sizes and
volume fractions in the heating/cooling cycles

| | | | After |
|---|---|---|---|
| PEG2000- | | | heating/cooling |
| PCL2000/PFP | Initial | Heating at 37° C. | cycle |
| concentration | Particle Size, nm/Volume Fraction, % | | |
| 1%/0.1% | 21/28; 91/20; | 26/46; 138/11; | 24/30; 104/26; |
| | 417/52 | 396/43 | 448/44 |
| 1%/1% | 161/44; | 36/81; 213/13; | 24/30; 104/26; |
| | 511/56* | 1054/6 | 401/45 |

*Droplet/bubble/droplet conversion in heating/cooling cycle is shown in bold fonts

In summary, micelles of 21-24 nm and a small fraction of larger associates (174 nm) are observed in the initial copolymer solution (no PFP). Upon adding PFP and sonicating at 20 kHz for 15 s, an emulsion is formed and the particle size distribution becomes tri-modal. The smallest particles (42 nm) are presumably micelles with PFP dissolved in the core; larger particles (274 nm and 1500 nm) are nano/microdroplets. All three system components are preserved upon a 45-fold dilution

With respect to dilution effect, although the sizes of the particle are somewhat decreased, the micelles and microdroplets are preserved upon a 45-fold dilution. Note that intravenous injections are associated with the injected system dilution by about 20-25-fold.

With respect to the effect of copolymer concentration: the higher copolymer concentration, the smaller nanodroplets (for the same [PFP]).

With respect to the effect of PFP concentration, micelles (20 nm) disappear at a high PFP concentration while larger associates or small nanodroplets (160 nm) are formed in addition to larger nanobubbles.

With respect to heating effect, upon heating, nanodroplets are converted into microbubbles; some larger microbubbles evaporate; copolymer molecules released due to bubble collapse self-assemble into micelles. Due to a disappearance of larger bubbles, upon cooling, mean droplet size is somewhat decreased (See, FIG. 7). Bubbles stabilized by PEG-PCL copolymer are more stable under heating than those stabilized by PEG-PLLA copolymer.

The following is a summary of ultrasound effects on the nanodroplet system:
1) Under pulsed 1-MHz ultrasound, micron-sized droplets are broken into smaller droplets, which require more copolymer for droplet stabilization; this leads to micelle (24 imn) disappearance. Under CW ultrasound, droplets are almost completely destroyed and gas evaporated; a released copolymer forms micelles.
2) Under 3-MHz ultrasound, even at a lower power density of 2.0 W/cm2 and a low duty cycle of 20%, the droplets are effectively converted into microbubbles that collapse leaving only micelles behind (compare FIGS. 14, d and e).

### Example 10

### Pluronic P-105 Stabilized Nanodroplets

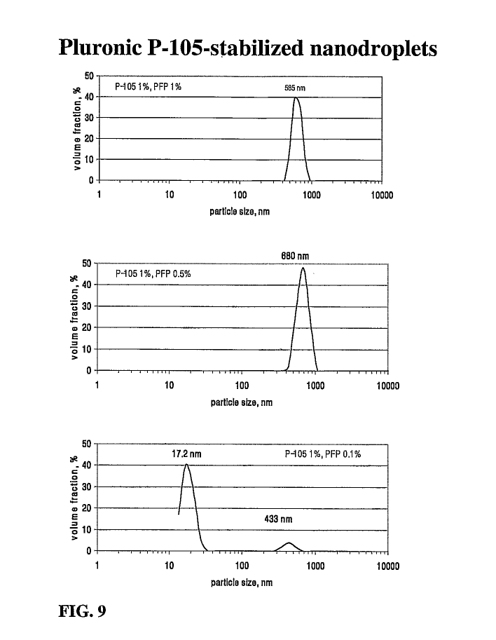

Size distribution was measured one day after the emulsion preparation; the system was kept at RT. Droplet sizes are smaller than those observed for PEG2000-PLLA2000. For Pluronic P-105, more copolymer is required for droplet stabilization; for Pluronic concentration of 1%, micelles are observed at a PFP concentration of 0.1% but not at a PFP concentration of 0.5% or higher, in contrast to a PEG2000-PLLA2000 and PEG2000-PCL2000, with a 0.5% PFP (See, FIG. 9). Stability upon heating was poor for Pluronic-stabilized systems and therefore, they were not further investigated.

### Example 11

### Drug Localization and Drug Retention

Fluorescence micrographs of the nano/microdroplets and microbubbles showed that anticancer drug Doxorubicin (DOX) is found localized on the copolymer-coated droplet or bubble surface (FIG. 15).

Drug retention in nano/microdroplets was measured by dialysis through a 7.2 kDa membrane; 5 ml of a DOX-loaded 0.5% PEG2000-PLLA2000, 2% PFP, 0.75 mg/ml DOX formulation was dialyzed against 200 ml PBS. The data showed that (i) DOX release from the above emulsion formulation was slightly faster than that from a micellar 0.5% PEG2000-PLLA2000, 0.75 mg/ml DOX formulation and (b) more than 90% of the drug was effectively retained in the emulsion.

### Example 12

### Drug Delivery to A2780 Ovarian Carcinoma Cells Using PEG2000-PLLA2000 Micelles/Microemulsions as Drug Carriers

Cells were grown in monolayers at 5% CO₂ humidified atmosphere in 6-well plates to 75% confluence. Before sonication, the growth media was replaced by DOX-loaded mixture of PEG2000-PLLA2000 micelles/microemulsions. A2780 cells were incubated for 5 minutes with 20 μg/ml DOX that was either dissolved in PBS or encapsulated in PEG2000-PLLA2000 micelles or a mixture of PEG2000-PLLA2000 micelles and PEG2000-PLLA2000/PFP microbubbles as described in Examples IV-XI. Microemulsions were formed in the 0.2% PEG2000-PLLA2000 micellar solution by adding 0.1% (v/v) PFP followed by mild sonication at 20 kHz for 15 seconds.

Ultrasound was unfocused and administered at 1 MHz or 3 MHz for 1 minute at a duty cycle of 33% (corresponding to 20 seconds of ultrasound exposure time) and power density of 3.4 W/cm², for 1 MHz, and 2.0 W/cm², for 3 MHz. An ultrasound transducer was attached to the bottom of the 6-well plate through an Aquasonic coupling gel.

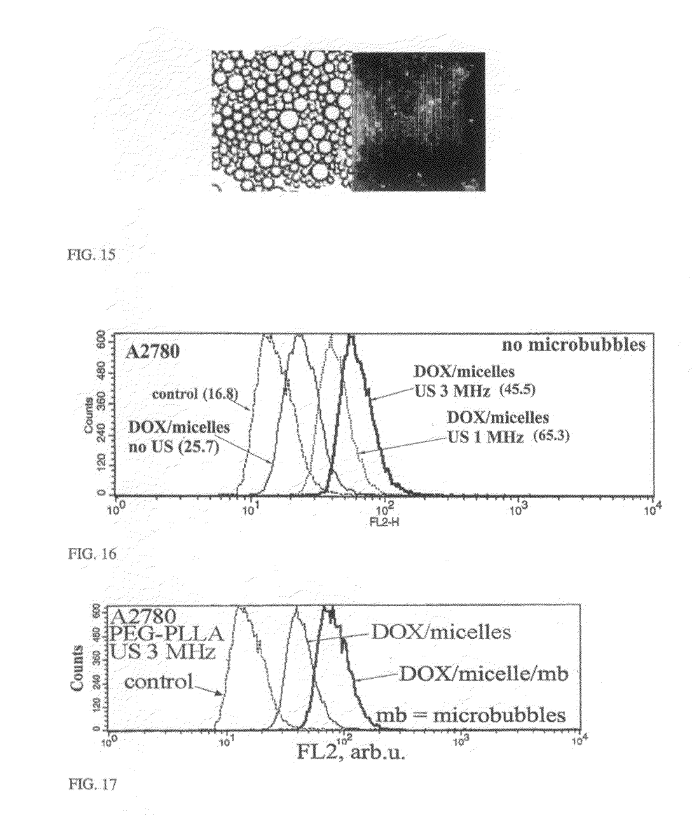

After sonication, cells were trypsinized and the DOX uptake and membrane damage was measured by flow cytometry. All experiments were repeated in triplicate. FIG. 16 shows the enhancing effect of ultrasound on the DOX delivery to the ovarian carcinoma A2780 cells from PEG-PLLA micelles in the absence of microbubbles. A significant enhancement of the intracellular drug uptake is observed. The dashed line represents the control (untreated A2780 cells) sample. The light solid line represents treatment of A2780 tumor cells with micelle encapsulated DOX, no ultrasound applied. The dotted line represents A2780 tumor cells treated with micelle encapsulated DOX, ultrasound was applied at 3 MHz. The solid dark line represents A2780 tumor cells treated with micelle encapsulated DOX, ultrasound was applied at 1 MHz.

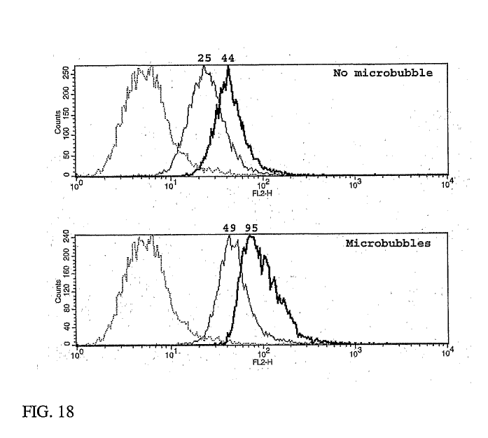

FIG. 17 shows the effect of microbubbles on the DOX delivery to ovarian carcinoma A2780 cells from PEG-PLLA micelle/microbubble system. The presence of microbubbles additionally enhances the intracellular uptake of DOX. The data composing the dashed line represents control (untreated) cells. The data composing the light solid line represents A2780 tumor cells treated with micelle encapsulated DOX (no microbubbles), ultrasound was applied at 3 MHz. The data composing the dark solid line represents A2780 tumor cells treated with micelle encapsulated DOX, ultrasound was applied at 3 MHz, and microbubbles were co-administered. The data indicate that treatment of the cells using the micelle/microbubble systems as disclosed herein, combined with ultrasonic irradiation at 3 MHz, results in an effective intracellular drug uptake.

The amount of ultrasonic irradiation to be applied against any tumor will depend on several variables such as the type of tissue the tumor originates from, location of the tumor or tumors in the subject, the size of tumor in thickness or width or any other dimension, the amount of drug or biologically active agent to be injected, and other environmental variables able to be determined by one of ordinary skill in the art.

### Example 13

### Effect of Microbubbles on Ultrasonic Drug Delivery to Tumor Cells In Vitro and In Vivo

### (a) DOX Targeting In Vivo to the MDA MB231 Breast Cancer Tumors

Note that in the absence of PFP, DOX is encapsulated in PEG2000-PLLA2000 micelles; in the presence of 2% PFP, no micelles are preserved (FIG. 8) since all the copolymer is used for droplet stabilization.

In the biodistribution experiments, DOX was injected intravenously to four MDA MB231 breast cancer tumor bearing mice. Two mice were injected with a micellar formulation (PEG2000-PLLA2000 0.5%, DOX 0.75 mg/ml); two other mice were injected with the emulsion formulation (micellar formulation supplemented with 2% PFP). Four hours after the drug injection, tumors of two mice were sonicated for 150 s by 3-MHz ultrasound at a power density of 2 W/cm2 and duty cycle of 20% (resulting in a 30-s ultrasound exposure). Ten minutes after sonication, all mice were sacrificed, tumors excised and trypsinized; the intracellular DOX fluorescence was measured by flow cytometry. The results are shown in Table 9.

TABLE 9
DOX intracellular uptake by the tumor cells upon the intravenous DOX
injections in a micellar or emulsion formulation, with and
without sonication.

| Formulation and protocol | Tumor Cell Fluorescence, arb.u. |
|---|---|
| Control | 11.3 |
| Micelles (no PFP, no ultrasound) | 12.2 |
| Microbubble (PFP, no ultrasound) | 15.4 |
| Micelle/ultrasound | 17.2 |
| Microbubble/ultrasound | 26.4 |

Table 9 shows that (i) tumor sonication in the presence of microbubbles results in a dramatic increase of the intracellular drug uptake compared to the drug delivery in the same formulation without ultrasound; and (ii) ultrasound effect on the intracellular drug uptake is much stronger in the presence of microbubbles than in micellar formulation. This confirms in vivo bubble preservation for four hours suggested by the data of FIGS. 7, 8.

### (b) Effect of Microbubbles and Ultrasound on the Intracellular Drug Uptake by the A2780 Ovarian Carcinoma Cells in Monolayers

These experiments were performed with various tumor cell lines; the cells were either in suspension or attached to the substrate in monolayers. The results for the ovarian carcinoma A2780 cells sonicated by focused ultrasound in suspensions are presented in Table 10. As follows from the data, in the absence of ultrasound, drug uptake from the emulsion was slightly higher than that from micelles, in accordance with DOX localization on the bubble surface and a higher release upon dialysis; under ultrasound, the enhancing effect of microbubbles on the intracellular DOX uptake was very dramatic: the ultrasound-modulated intracellular DOX uptake by the ovarian carcinoma cells doubled in the presence of microbubbles. The effect of microbubble and ultrasound on the intracellular DOX uptake by A2780 cells is shown in FIG. 17.

### (c) Effect of Microbubbles and Ultrasound on the Intracellular Drug Uptake by the Cells of the Excised MDA MB231 Breast Cancer Tumors

A new technique was developed to study the effect of microbubbles and ultrasound on the intracellular drug uptake by the cells of the excised tumors. The same PEG-PLLA/PFP/DOX formulation as that described for Table 10 was used in these experiments. The results for the breast cancer MDA MB231 tumor are shown in Table 11 and FIGS. 18 A and 18 B.

TABLE 10
Effect of microbubbles and ultrasound on the intracellular DOX
uptake by the ovarian carcinoma A2780 cells in suspensions.
Formulation: PEG2000-PLLA2000 0.5%; PFP 2%, DOX = 0.75 mg/ml.
Ultrasound parameters: focused ultrasound, frequency 1.1 MHz,
pressure amplitude 0.55 MPa, duty cycle 33%, duration 15 s.
Measurements are based on the intracellular DOX fluorescence.

| Drug delivery conditions | Cell Fluorescence, arb.u. |
|---|---|
| Control | 13 |
| No microbubble, no ultrasound | 72 |
| No microbubble, ultrasound | 68 |
| Microbubbles, no ultrasound | 89 |
| Microbubbles, ultrasound | 140 |

TABLE 11
Effect of microbubbles and ultrasound on the intracellular DOX
uptake by the cells of the freshly excised MDA MB 231 tumor.
Formulation: [DOX] = 0.75 mg/ml; [PEG-PLLA] = 0.5%; [PEP]
(if any) = 2%. Ultrasound parameters: unfocused ultrasound, frequency
3 MHz, power density 2 W/cm², duty cycle 50%, duration 1 min.
Measurement is based on the intracellular DOX fluorescence.

| Drug delivery conditions | Cell Fluorescence, arb u. |
|---|---|
| Control | 6 |
| No microbubble, no ultrasound | 25 |
| No microbubble, ultrasound | 44 |
| Microbubbles, no ultrasound | 49 |
| Microbubbles, ultrasound | 95 |

### (d) Drug Delivery to Multidrug Resistant (MDR) Ovarian Carcinoma A2780/AD Cells Using DOX-Loaded PEG2000-PLLA2000 Micelles/Microemulsions and Unfocused Ultrasound

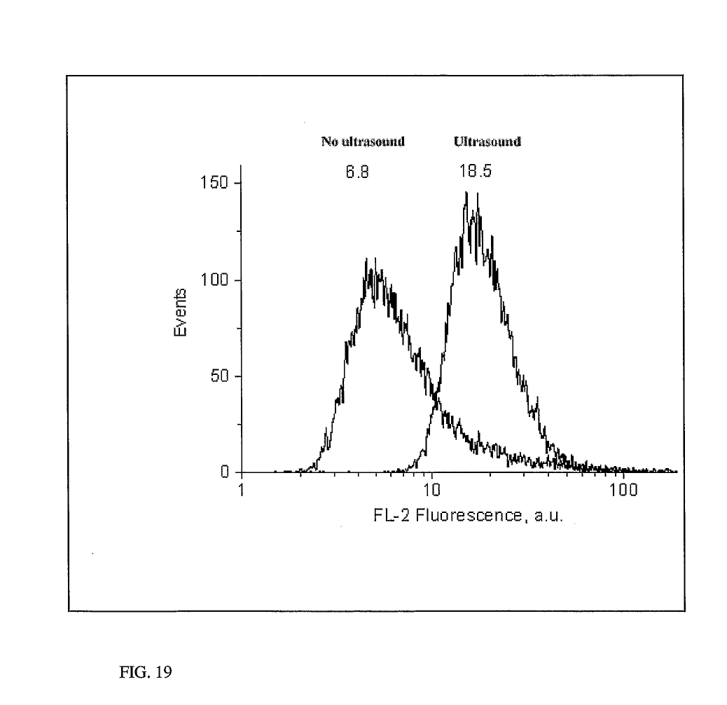

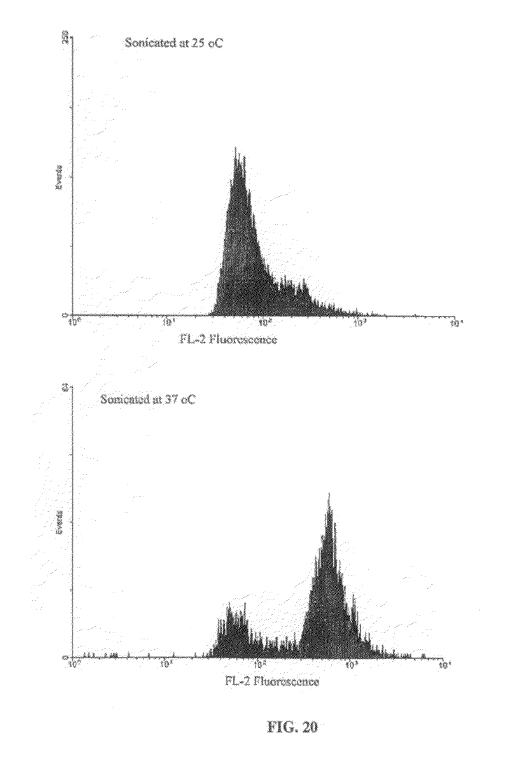

FIG. 19 shows the response to treatment of the MDR A2780/AD cells. The histograms of DOX fluorescence intensity in A2780/AD cells are shown for: (A) unsonicated cells treated with DOX dissolved in PBS (free DOX), and (B) cells sonicated for 1 minute at 1.0 MHz in the presence of DOX-loaded PEG2000-PLLA2000 micelles and microbubbles. (See, FIG. 19). The concentration of DOX was 20 μg/ml for both samples. Cells were incubated with DOX for 5 minutes prior to sonication. Sonication was administered for 1 minute by unfocused ultrasound at 33% duty cycle, 3.4 W/cm². A very significant enhancement of the intracellular drug uptake by the multidrug resistant cells was observed when the micelle/microbubble systems as disclosed herein are used. Furthermore, ultrasound treated samples, compared to untreated samples, further enhanced drug uptake.

### Example 14

### Drug Delivery to the MDR Ovarian Carcinoma A2780/AD Cells in Cultures Using DOX-Loaded PEG2000-PLLA2000 Micelles/Microemulsions and Focused Ultrasound at 25° C. and 37° C.

FIG. 20 shows the fluorescence histograms of the multidrug resistant ovarian carcinoma A2780/AD cells after 0.5 second sonication using focused 1.1 MHz ultrasound (33% duty cycle, 0.5 second pulse, 6 MPa pressure) in the presence of microemulsion. In the cells represented in FIG. 20A, sonication was performed at 25° C. In FIG. 20B cells were sonicated at 37° C. As is readily apparent from FIG. 20, a very significant enhancement of intracellular drug uptake is observed upon sonication at 37° C., suggesting that the echogenic properties of the microbubbles systems disclosed herein are more pronounced than those of the microemulsion systems disclosed herein under these conditions, in treating these tumor cells.

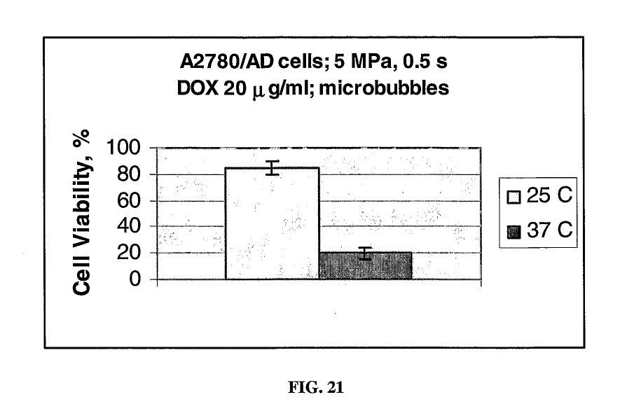

FIG. 21 shows the viability of multidrug resistant ovarian carcinoma A2780/AD cells after 0.5 second sonication by 1.1 MHz ultrasound (33% duty cycle, 0.5 second pulse length, 5 MPa pressure) in the presence of the microemulsion at either 25° C. or 37° C. Microbubble systems as disclosed herein dramatically enhanced the ultrasound-induced eradication of multidrug resistant ovarian carcinoma cells.

### Example 15

### Treatment of A2780 Tumors In Vivo

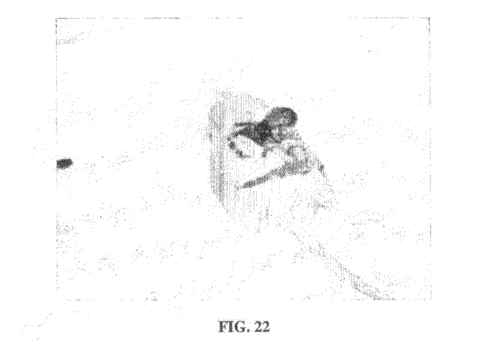

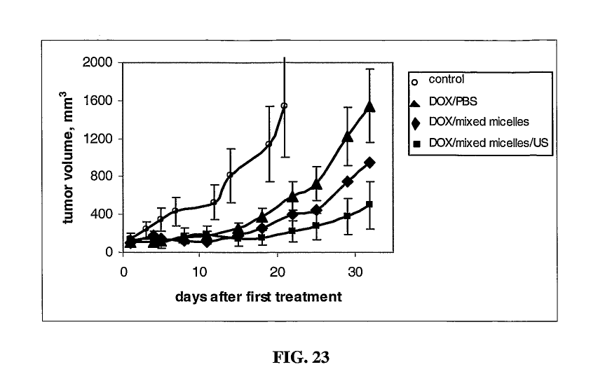

Tumors: A2780 ovarian cancer tumor in a female nu/nu mouse. This tumor is characterized in being more highly vascularized. (See, FIG. 22).

Polymeric Micelles: Polymeric Micelles: Micelles were formed using PEO/PPO/PEO triblock copolymer Pluronic P-105 (PEO/PPO/PEO ratio of 37/56/37) mixed with diacylphospholipid PEG2000-DSPE, (1:1 weight). (See, Z-G. Gao, et al., Controlled and Targeted Tumor Chemotherapy by Micellar-Encapsulated Drug and Ultrasound, J. Control. Release, 102:203-222, 2005). DOX was loaded into mixed micelles using a solvent evaporation technique as follows. Micelle-forming components and DOX were dissolved in chloroform and the solvent was evaporated. DOX-loaded micelles were then formed spontaneously upon addition of PBS to the dry residue. DOX-loaded micelles were injected intravenously (i.v.) at a DOX dosage of 3 mg/kg (growth rate studies) or 6 mg/kg (biodistribution studies).

Ultrasound: Unfocused ultrasound of 1 MHz, 3.4 W/cm² power density and 50% duty cycle was applied for 30 seconds, locally, to the tumor four hours after the drug injection.

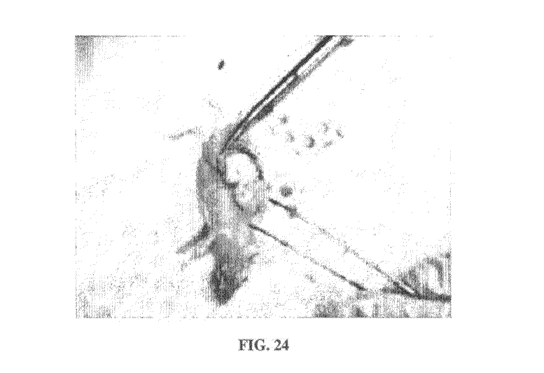

Results: A very significant inhibition of the tumor growth was observed for micelle/ultrasound technique. (See, FIG. 23). A significant drug accumulation was observed specifically in the tumor cells upon i.v. drug injection. (See, Z-G. Gao, et al., Controlled and Targeted Tumor Chemotherapy by Micellar-Encapsulated Drug and Ultrasound, J. Control. Release, 102:203-222, 2005). The effective treatment of A2780 tumors by i.v. injections is likely due to the extensive tumor vascularization, as seen in FIG. 22.

### Example 16

### Treatment of Poorly Vascularized HCT116 Colon Cancer Tumors

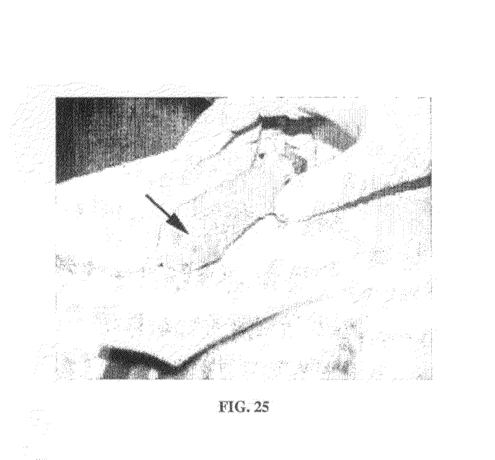

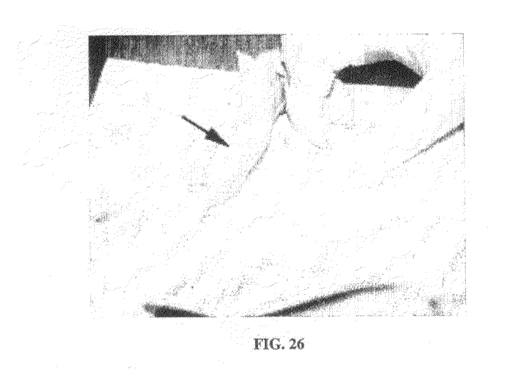

Tumors: HCT116 colon cancer tumors were inoculated subcutaneously to the right flanks of male nu/nu mice. (See, FIGS. 24 and 25). In contrast to A2780 tumors, this tumor is characterized as being poorly vascularized (See, FIG. 24).

Polymeric Micelles: DOX-loaded mixed PLURONIC® P-105/PEG2000-DSPE micelles (See, Gao et al., J. Control. Release 102 (2005) 203-221) were used in treatment of the tumors.

DOX-loaded micelles were injected either intravenously (i.v.) or intratumorally (i.t.) at a DOX dosage of 3 mg/kg (growth rate studies) or 6 mg/kg (biodistribution studies).

Ultrasound: Unfocused ultrasound of 1 MHz, 3.4 W/cm² power density and 33% duty cycle was applied for 30 seconds, locally, to the tumor four hours after the drug injection.

Biodistribution studies: DOX uptake by the cells of various organs was measured by flow cytometry. Mice were intratumorally injected with either free (i.e., PBS-dissolved) DOX or micelle-encapsulated DOX. Ten hours after injection of the composition, mice were sacrificed and tumors and various organs were excised and digested in trypsin. Individual cells were separated, fixed by 2.5% glutaraldehyde and analyzed by flow cytometry.

Tumor growth rates: Animals were randomly assigned to experimental and control groups, comprising five animals each. Intravenous treatment was initiated when tumor volume reached 50-100 mm³. Intratumoral treatment was initiated when tumor volume reached 150-300 mm³. A total of four injections were delivered twice a week for two weeks. For intratumoral injections, four injections of 25 μl drug solution each were made into four sites of a tumor.

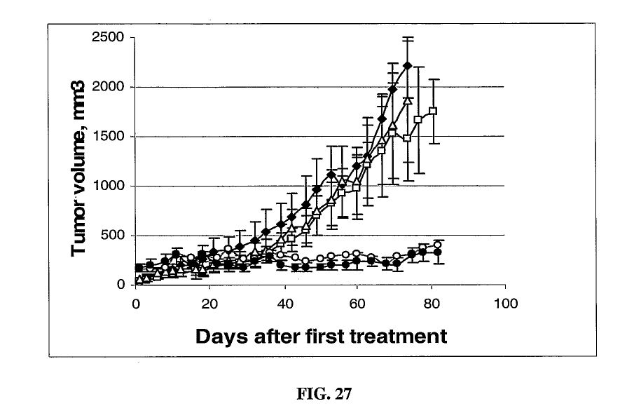

Results: Despite a moderate sensitivity to DOX (IC₅₀=1.5 μg/ml), the colon cancer HCT116 tumor manifested a high resistance to chemotherapy upon intravenous drug injection, independent of drug formulation (DOX dissolved in PBS or encapsulated in polymeric micelles). The resistance of the HCT116 tumor was likely caused by a poor vascularization resulting in insufficient drug delivery to tumor. Loose blood capillaries were located predominantly on the tumor surface in the HCT116 tumors, with the interior of the tumor having almost no blood supply and manifesting necrotic tissue and ascetic fluid. For this poorly vascularized tumor, a successful treatment was achieved by intratumoral injections of DOX encapsulated in polymeric micelles. (See, FIGS. 26 and 27).

FIG. 27 illustrates growth curves of HCT116 tumors upon intravenous Upper set of curves) and intratumoral (lower set of curves) injection of DOX. Solid diamonds represent intravenous administration control samples. Open squares represent DOX/PBS intravenous treatment. Open triangles represent micelle encapsulated DOX intravenous treatment followed by ultrasound. Open circles represent micelle encapsulated DOX intratumoral treatment. Closed circles represent micelle encapsulated DOX intratumoral treatment followed by ultrasound. Dramatic differences in the effects of the intravenous versus intratumoral injections are observed on the growth of HCT116 colon cancer tumors. The intratumoral DOX injections resulted in arrest of tumor growth. Local ultrasonic irradiation of the tumor additionally enhanced the effect of intratumoral chemotherapy resulting in a reduction of tumor volume and in some instances, a complete tumor resolution (See, FIG. 27).

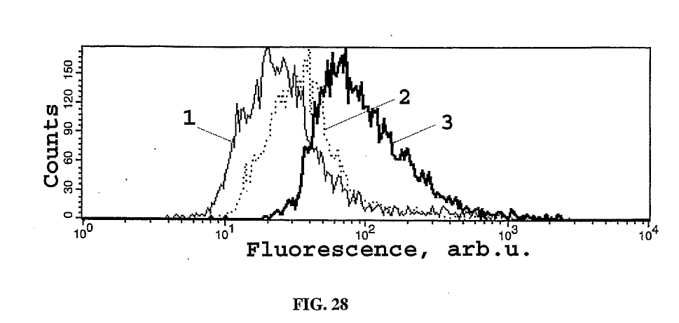

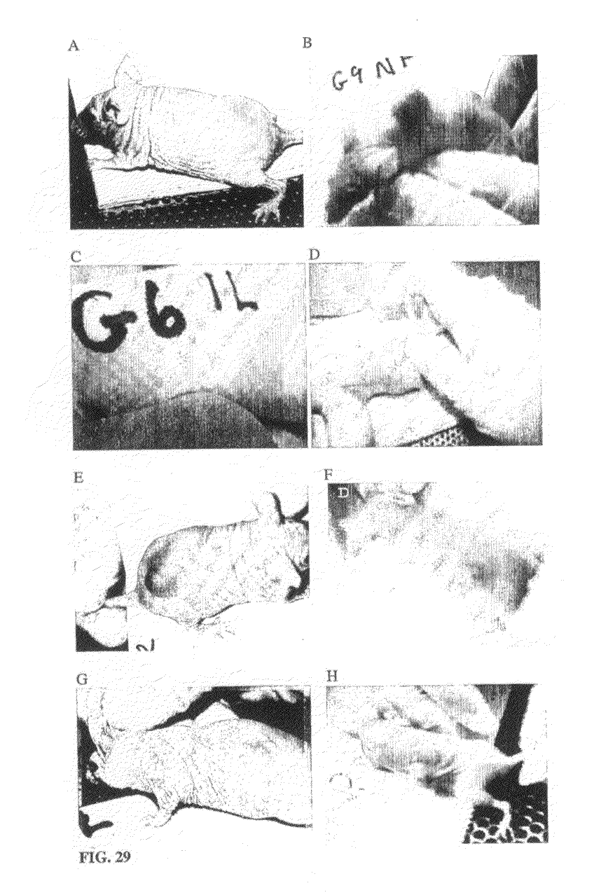

The biodistribution studies showed that polymeric micelle-encapsulated DOX was retained in the tumor tissue much longer than DOX dissolved in PBS. (See, FIG. 28). FIG. 28 shows fluorescence histograms of the tumor cells for free or micelle-encapsulated DOX upon intratumoral injections. The thin line represents the control, the dotted line represents DOX solubilized in PBS (free DOX), and the thick line represents micelle-encapsulated DOX.

Ten hours after intratumoral injection, micelle-encapsulated DOX was retained in the tumor cells much more effectively than free DOX (i.e. DOX dissolved in PBS). No DOX uptake by the heart cells was observed for micelle-encapsulated DOX while significant uptake of DOX was observed in the heart cells of animals treated with PBS-dissolved DOX (data not shown). Such non-target tissue drug uptake causes DOX cardiotoxicity. Interestingly, no DOX was found in the liver and kidney cells in animals treated with encapsulated DOX. However, in these animals, some DOX was observed in spleen cells. The intracellular concentration of DOX in the spleen cells was slightly higher in animals treated with free DOX as compared to animals treated with micelle-encapsulated DOX.

In animals given i.v. injections of the compositions, neither free nor micelle-encapsulated DOX was noticeably internalized by the tumor cells ten hours after the injection. Thus, intravenous injections did not deliver drug to tumor cells. In contrast to intravenous injections, intratumoral DOX injections effectively delivered drugs to target tumor cells. Micelle-encapsulated DOX was more efficient than free DOX as exhibited by the longer tumor retention time and a preferential localization in the tumor cells.

### Example 17

### Effect of Microbubbles and Ultrasound on the MDA MB231 Tumor Growth In Vivo

Microbubble suspensions injected either intravenously, subcutaneously, or intratumorally to nu/nu mice did not cause any adverse effects. When injected subcutaneously or intratumorally, the microbubbles remained at the site of injection for several weeks and therefore could serve as a drug depot delivering drug on demand under the action of ultrasonic stimuli; the “bump” formed at the injection site gradually decreased and completely disappeared by the end of the month, most probably due to the biodegradation of a stabilizing copolymer.

The MDA MB231 tumors were inoculated in nu/nu mice. Overall of eight treatment groups were studied in this experiment:

1—control
2—intravenous (iv) injections of a micellar formulation (no PFP, no ultrasound)
3—iv injections of a microbubble formulation (no PFP, no ultrasound)
4—intratumoral (it) injections of a micellar formulation (no PFP, no ultrasound)
5—it injection of a microbubble formulation (no PFP, no ultrasound)—one treatment
6—iv injections of a micellar formulation, ultrasound
7—iv injections of a microbubble formulation, ultrasound
8—it injections of a microbubble formulation, ultrasound—one treatment

Mice were treated by 3 mg/kg DOX encapsulated in PEG2000-PLLA2000 (0.5% polymer in a PBS solution) formulated with 2% (vol.) PFP (denoted as microbubble formulation). Some mice were sonicated for 150 s by unfocused 3-MHz ultrasound at 2 W/cm² power density and 20% duty cycle. Ultrasound was applied locally to the tumor four hours after the intravenous drug injections or 15 min after the direct intratumoral injections. Four treatments by the intravenous injections were applied, with a 3-day break between the treatments (groups 2, 3, 6, and 7). The same regiment was applied for the intratumoral injections of a micellar formulation (group 4). Only one intratumoral treatment by a microbubble formulation was applied for groups 5 and 8.

It should be noted that upon intravenous or intratumoral injections of the microbubble formulation without ultrasound, there was no effect of drug-loaded microbubbles on the tumor growth suggesting a significant degree of drug retention by the microbubbles. However a dramatic arrest of the tumor growth was observed when the intravenous (compare D and C) or intratumoral (compare F and E or H and G) injections of the microbubble formulation were combined with sonication by 3-MHz ultrasound. The photographs of the tumors before and a month after the treatment are shown in FIG. 29. Treatment protocols and tumor ages at the date of shooting are shown in Table 12.

TABLE 12
Panel codes, treatment protocols and tumor ages for mice inoculated
with breast cancer MDA MB231 tumors, as depicted in FIG. 29. Mice
were treated by 3 mg/kg DOX encapsulated in PEG2000-PLLA2000
(0.5% polymer in a PBS solution) formulated with 2% (vol) PFP
(denoted as microbubble formulation). Some mice were sonicated for
150 s by unfocused 3-MHz ultrasound at 2 W/cm² and 20% duty cycle.

| | | Tumor age, |
|---|---|---|
| Panel code | Treatment protocol | days |
| A | control | 50 |
| B | control | 79 |
| C - before the start of the | | 30 days |
| intravenous treatment | | |
| D - mouse C, 30 days after | Four treatments within two | 60 days |
| the intravenous treatment | weeks by the intravenous | |
| | injections of a microbubble | |
| | formulation combined with | |
| | tumor sonication | |
| E - before the start of the | | 50 |
| intratumoral treatment | | |
| F - mouse E 29 days after the | One treatment by a direct | 79 |
| treatment | intratumoral injections of a | |
| | microbubble formulation | |
| | combined with tumor | |
| | sonication | |
| G - before the start of the | | 50 |
| intratumoral treatment | | |
| H - mouse E 43 days after the | One treatment by a direct | 93 |
| treatment | intratumoral injections | |
| | combined with tumor | |
| | sonication | |

As manifested by the photographs, the intravenous and intratumoral injection of a microbubble formulation combined with ultrasonic tumor treatment were effective in the arrest of the tumor growth and disease stabilization. However without ultrasound, the intravenous injections of the microbubble formulation were less effective than the injections of micellar formulations. Also, without ultrasound, the intratumoral injections of the microbubble formulation were not effective in preventing tumor growth.

Without being bound by a theory of the invention, the following might help explain the good results described herein.

1. Without ultrasound, the intravenous injections of a microbubble formulation are less effective than those of a micellar formulation. The possible cause of this effect is that drug-loaded micelles accumulate in the tumor volume while large microbubbles do not penetrate through the blood capillaries and do not target drug to the tumor unless sonicated.
2. Tumor sonication enhanced the action of both, micellar and microbubble formulation. In the presence of microbubbles, the growth of sonicated tumors was completely inhibited (group 7).
3. Even one-time intratumoral injection combined with tumor sonication effectively prevented tumor growth and resulted in disease stabilization. These data are illustrated in the photographs of FIG. 29.

### Example 18

### Effect of the Intravenous and Intratumoral Injections of Micellar-Encapsulated Paclitaxel Combined with Tumor Sonication on the Growth of Multidrug Resistant Breast Cancer MCF7/AD Tumors

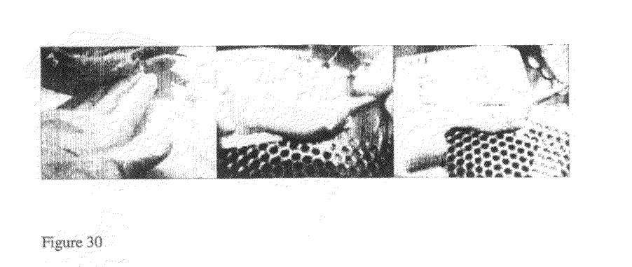

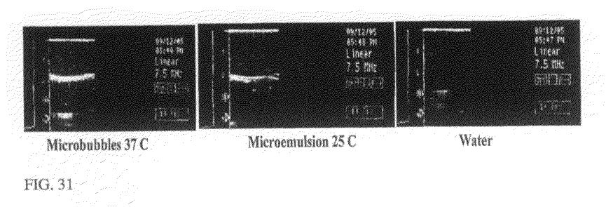

Without ultrasound, these tumors were very resistant to the treatment by either clinical paclitaxel formulation (Paclitaxel Injections) or micellar formulation (paclitaxel encapsulated in the mPEG-PDLLA micelles; the latter formulation was developed and donated by Samyang Pharmaceuticals, Korea). However with the application of ultrasound, a significant suppression of the tumor growth was observed; in 80% cases, tumors were completely resolved. This was true for both intravenous and intratumoral drug injections. As an example, FIG. 30 shows the photographs of a mouse treated by the intratumoral injections of micellar-encapsulated paclitaxel combined with tumor sonication. Only the left of the two tumors shown in FIG. 30 was treated by the direct injection, the second tumor was used as a control. The treated tumor was completely resolved a month and a half after the end of the treatment indicating a high efficiency of the ultrasound-enhanced intratumoral chemotherapy of multidrug resistant tumors.

### Example 19

### Ultrasound Imaging

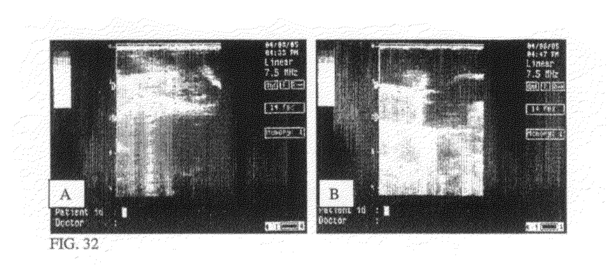

(a) The ultrasound images at 7.5 MHz of phantom capillaries filled with water, nanoemulsion (at RT), or nano/microbubble (at 37° C.) are shown in FIG. 31 for PEG2000-PCL2000 1%-1% system. Both nanoemulsion and nano/microbubbles proved echogenic; however microbubbles manifested a higher ultrasound contrast than nanodroplets.

(b) The ultrasound images at 7.5 MHz of empty or drug-loaded microbubbles described in Example 9 (PEG2000-PLLA2000 concentration of 0.2%, PFP concentration of 0.1%) injected into the beef liver maintained at room temperature (left) or agarose tissue phantom preheated to 37° C. (right). Images were comparable to those obtained with Optison® microbubbles in parallel runs.

While this invention has been described in certain embodiments, the present invention can be further modified within the spirit and scope of this disclosure. The invention may be used with any drug carrier, the structure of which may be disrupted under the action of microbubble-enhanced ultrasound thus locally releasing the drug load in the sonicated tissue. For example, energy disruption techniques, such as ultrasound radiation, enhance drug release or delivery by liposomes. Techniques for ultrasound-assisted drug release by liposomes are reported by inventors at the seminar in the Radiology Department of the National Institutes of Health's Clinical Center in August, 2005, and also reported in the Science magazine (Harder, B. “Ultrasound's New Focus, can it eradicate tumors”, Science, Apr. 29, 2006; Vol. 169, No. 17, p 264). The contents of the two reports are incorporated herein by this reference. The list of possible drug carriers that can be used with the microbubbles includes but is not limited to micelles, liposomes, nanoemulsions, microemulsions, nanobubbles, nanoshell particles, etc. Techniques of using micelles, liposomes, nanoemulsions, microemulsions, nanobubbles and nanoshell particles as drug carriers are known in the art of drug delivery.

This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

What is claimed is:

1. A method of treating a tumor, said method comprising:
injecting into the tumor an agent, wherein said agent is encapsulated in a mixture of a drug carrier and a microemulsion.

2. The method according to claim 1, wherein the tumor is a poorly vascularized tumor.

3. The method according to claim 1, wherein the tumor is a multidrug resistant tumor.

4. The method according to claim 1, wherein the tumor is an inoperable tumor.

5. The method according to claim 1, said method further comprising:
applying a means for disrupting the drug carrier and microemulsion at the tumor site after injection of said drug carrier and microemulsion.

6. The method according to claim 5, wherein said means for disrupting the drug carrier and microemulsion comprises ultrasonic radiation.

7. The method according to claim 1, wherein said microemulsion forms microbubbles in situ upon injection.

8. The method according to claim 1, wherein said agent is selected from one or more of the group of biologically active agents consisting of doxorubicin, adriamycin, cisplatin, taxol, 5-fluorouracil, betulinic acid, amphotericin B, diazepam, nystatin, propofol, testosterone, estrogen, prednisolone, prednisone, 2,3-mercaptopropanol, progesterone, and mixtures of any thereof.

9. A method of treating a tumor, said method comprising:
injecting intravenously an agent capable of treating a tumor, said agent being encapsulated in a mixture of a drug carrier and a microemulsion.

10. The method according to claim 9, wherein said microemulsion forms microbubbles in situ upon injection.

11. A method of treating a tumor, said method comprising
preparing a microemulsion capable of transforming into microbubbles upon injection into a tissue;
preparing a drug carrier;
encapsulating at least one agent in the drug carrier;
mixing the microemulsion with the drug carrier to form a composition; and
injecting the composition into or near at least one tumor in a subject, thus forming the microbubbles and treating said tumor.

12. The method according to claim 11, further comprising:
applying a means of disrupting the drug carrier and microbubbles at the site of the at least one tumor.

13. The method according to claim 12, wherein said means of disrupting the drug carrier and microbubbles comprises ultrasonic radiation.

14. The method according to claim 13, wherein said ultrasonic radiation is administered from about 100 kHz to about 10 MHz.

15. The method according to claim 13, wherein said ultrasonic radiation is administered from about 0.5 to about 7 MPa negative pressure.

16. The method according to claim 11, wherein said drug carrier is polymeric micelles formed from poly(ethylene oxide)-poly(propylene oxide)-poly(ethylene oxide) copolymer.

17. The method according to claim 11, wherein said drug carrier is polymeric micelles formed from a diblock copolymer of poly(ethylene glycol)-co-poly(L-lactide).

18. The method according to claim 11, wherein said drug carrier is polymeric micelles formed from a diblock copolymer of poly(ethylene glycol)-co-poly(caprolactone).

19. The method according to claim 11, wherein said microemulsion is composed of perfluoropentane and at least one copolymer selected from the group consisting of poly(ethylene glycol)-co-poly(L-lactide), poly(ethylene glycol)-co-poly(D,L-lactide), poly(ethylene glycol)-co-poly(caprolactone), and poly(ethylene oxide)-poly(propylene oxide)-poly(ethylene oxide) copolymer.

20. A method of directing an agent into a tissue, said method comprising:
injecting a microemulsion into a tissue or a circulation, wherein said microemulsion comprises perfluoropentane and a drug carrier and forms microbubbles in situ upon injection, and further wherein said microemulsion also comprises an agent; and
visualizing said microemulsion using an ultrasound imager.

21. The method according to claim 20 or 25, wherein said agent is also encapsulated in the drug carrier.

22. A method of enhancing delivery of an agent to a cell, said method comprising:
preparing a drug carrier;
preparing a microemulsion capable of transforming into microbubbles upon injection into a tissue comprising at least one cell;
encapsulating at least one agent in the drug carrier,
mixing the microemulsion with the drug carrier to form a composition; and
injecting the composition into a tissue, and
applying a means for disrupting the drug carrier and microemulsion at the tissue such that said means of disrupting the drug carrier and microemulsion enhances delivery of said agent to the at least one cell.

23. The method according to claim 22, wherein said means of disrupting the drug carrier and microemulsions comprises ultrasonic radiation.

24. The method according to claim 1, 9, 11, 20, 22 or 27, wherein said microemulsion comprises at least one perfluorocarbon compound.

25. A method of directing an agent into a tissue, said method comprising: injecting a microemulsion into a tissue, wherein said microemulsion comprises at least one perfluorocarbon compound and a drug carrier and forms microbubbles in situ upon injection, and further wherein said microemulsion also comprises an agent; and visualizing said microemulsion using an ultrasound imager.

26. The method according to claim 1, 9, 11, 20, 22, 25 or 27, wherein said microemulsion comprises nanoparticles.

27. A method of treating a tumor, said method comprising
preparing a microemulsion capable of transforming into microbubbles upon injection into a tissue;
preparing a drug carrier;
encapsulating at least one agent into the mixture of the drug carrier and microemulsions and form a composition;
injecting the composition into or near at least one tumor in a subject, thus forming the microbubbles and treating said tumor.

28. The method according to claim 1, 9, 11, 20, 22, 25 or 27, wherein said microemulsion and/or said drug carrier comprises at least one enhancing agent as stabilizer.

29. The method according to claim 11, 22 or 27, wherein said composition is sterile.

30. The method according to claim 11, wherein said drug carrier is copolymer micelles formed from a diblock copolymer of poly(ethylene glycol)-co-poly(D,L-lactide).

31. The method according to claim 6, 13 or 23, wherein the ultrasonic radiation is applied extracorporeally, intraluminally, or interstitially

32. The method according to claim 20 or 25, wherein the ultrasound imager is applied extracorporeally, intraluminally, or interstitially.

33. The method according to claim 1, 9, 11, 20, 22, 25 or 27, wherein the drug carrier is selected from the group consisting of polymeric micelles, micelles, liposomes, nanoemulsions, microemulsions, nanoshell particles and any combinations thereof.
